(19) United States
(12) Patent Application Publication
(10) Pub. No.: US 2018/0193443 A1
(43) Pub. Date: Jul. 12, 2018

(54) CAMPYLOBACTER JEJUNI PEPTIDE FOR VACCINE DEVELOPMENT

(71) Applicant: Iowa State University Research Foundation, Inc., Ames, IA (US)

(72) Inventors: Qijing Zhang, Ames, IA (US); Zuowei Wu, Ames, IA (US); Orhan Sahin, Ames, IA (US); Michael Yaeger, Ames, IA (US); Paul Plummer, Ames, IA (US)

(21) Appl. No.: 15/800,770

(22) Filed: Nov. 1, 2017

Related U.S. Application Data
(60) Provisional application No. 62/415,750, filed on Nov. 1, 2016.

(51) Int. Cl. A61K 39/02; C07K 14/205

(52) U.S. Cl. CPC A61K 39/105; A61K 2039/552; C07K 14/205; A61K 39/0208

(57) ABSTRACT

Immunogenic compositions directed against C. jejuni, particularly hypervirulent strains thereof that are able to translocate the intestinal tract and cause systemic infection and abortion, are provided. Applicants have identified a novel major outer membrane protein and particularly a peptide portion thereof that is indicative of the recent hypervirulent strains and may be used in vaccine protocols and immunogenic and diagnostic compositions.

### CROSS REFERENCE TO RELATED APPLICATION

This application claims priority under 35 U.S.C § 119 to Provisional Patent Application Ser. No. 62/415,750 filed Nov. 1, 2016, herein incorporated by reference in its entirety.

### GRANT REFERENCE

This invention was made with government support under Grant Nos. 2013-67015-20368, awarded by USDA/NIFA. The government has certain rights in the invention.

### FIELD OF THE INVENTION

The present invention relates to immunogenic and vaccine compositions comprising a peptide in the major outer membrane peptide from Campylobacter jejuni and to methods of use relating thereto.

### BACKGROUND OF THE INVENTION

Campylobacter jejuni is a major foodborne pathogen and a leading cause of enteritis in humans, responsible for 400-500 million cases of diarrhea annually worldwide. In the United States alone, Campylobacter accounts for more than 800,000 cases of foodborne illnesses each year. As a zoonotic pathogen, C. jejuni is widely distributed in the gut microbiota of wild and domesticated animal species, such as cattle, sheep, and poultry. Transmission of C. jejuni to humans is mainly via con-taminated meat, milk, and water. Although C. jejuni is primarily a gut colonizer, some hypervirulent strains may be able to translocate across intestinal epithelium, producing bacteremia and systemic infections. In addition to causing foodborne illnesses, C. jejuni is also a primary etiological agent for ruminant abortion. Recently, it has been reported that there is an emergence of an antibiotic-resistant and hypervirulent clone of C. jejuni in the United States. It is named clone “sheep abortion” (SA) and is responsible for the majority (>90%) of ovine abortion cases in the United States. The clonality of SA strains was established by multiple molecular typing methods, including pulsed-field gel electrophoresis (PFGE) and multilocus sequence typing (MLST) (ST-8). Notably, C. jejuni clone SA is zoonotic and has been implicated in a number of outbreaks and sporadic cases of foodborne illnesses in humans. Clone SA infection is characterized by its extraordinary ability to translocate across the intestine, induce systemic infection, invade the uteroplacental unit with high titer, and cause abortion in pregnant animals compared with other strains of C. jejuni. A recent study revealed that the genetic basis for its enhanced virulence is due to specific mutations in the major outer membrane protein encoded by the porA gene.

### SUMMARY OF THE INVENTION

The invention encompasses an immunogenic composition. Applicants have identified a peptide sequence in the major outer membrane protein (MOMP) of C. jejuni that is essential for its hyper-virulence in inducing abortion. The peptide sequence (AEEQGADLLGKSTISTTQKAAPFQADSLGNL) (SEQ ID NO: 1) is located in the predicted external loop 4 of MOMP encoded by porA. The loop 4 sequence is critical for virulence and provides a target for designing a diagnostic test (for identifying this hypervirulent strain) and for vaccine development to control C. jejuni infections. The immunogenic composition or vaccine comprises in one embodiment C. jejuni hyper-virulence peptide sequence from the major outer membrane protein and one or more excipients in an amount effective to elicit production of neutralizing antibodies against the C. jejuni pathogen following administration to a subject. In some embodiments, the immunogenic composition comprises one or more additional components such as another antigen in Campylobacter adjuvants or excipients. In some emobiemetnst the peptide includes one or more amino acid modifications of SEQ ID NO:1.

The invention also encompasses a method of producing a protective immune response against the hyper-virulent C. jejuni strain in a subject comprising administering to the subject the immunogenic composition described herein in an amount and duration effective to produce the protective immune response. In some embodiments, the protective immune response reduces clinical disease and/or hyper-virulent C. jejuni reproduction in the subject and may also reduce hyper-virulent C. jejuni shedding in the subject. In some embodiments, the subject has been exposed to hyper-virulent C. jejuni while in other embodiments, the subject is suffering from a hyper-virulent C. jejuni infection. In some embodiments, the invention encompasses a method of producing a protective immune response against hyper-virulent C. jejuni in a subject comprising administering to the subject the immunogenic composition described herein in an amount and duration effective to produce the protective immune response. In some embodiments, the invention encompasses a method of producing a protective immune response against hyper-virulent C. jejuni in a subject comprising administering to the subject the immunogenic composition described herein in an amount and duration effective to produce the protective immune response.

The invention further encompasses a method of differentiating a subject vaccinated with the immunogenic composition described herein from a subject exposed to hyper-virulent C. jejuni comprising detecting the presence of an antibody in a biological sample isolated from the subject against the C. jejuni major outer membrane protein. The immunogenic compositions and methods of the invention can be administered to a subject such as a human, cow, sheep, goat, or chicken.

The invention also encompasses a method of producing a protective immune response against hyper-virulent C. jejuni in a human subject comprising administering to the subject an immunogenic composition comprising the MOMP peptide in an amount and duration effective to produce the neutralizing antibody response. In some embodiments, the immunogenic composition further comprises an adjuvant.

The present invention provides a vaccine composition for protecting animals against C. jejuni, including a highly virulent C jejuni strain, the composition including a C. jejuni MOMP peptide, wherein the polypeptide comprises SEQ ID NO: 1 or a variant thereof. In one embodiment, the composition also provides heterologous protection against other C. jejuni strains.

In one embodiment, the composition is in the form of an inactivated, C. jejuni whole cells that comprises and/or expresses the hyper-virulence associated peptide.

In another embodiment, the composition is in the form of an inactivated vector bacterium (such as E. coli), wherein the vector bacterium comprises and/or expresses the hyper-virulent C. jejuni MOMP polypeptide.

In yet another embodiment, the composition is in the form of an isolated, recombinant hyper-virulent C. jejuni MOMP polypeptide. In one embodiment, the isolated, recombinant C. jejuni MOMP polypeptide is expressed from a cloning vector. In another embodiment, the vector is an E. coli bacterium. In a further embodiment, the vector is a live or inactivated vector. In some embodiments, the composition of the present invention further includes an adjuvant. In one embodiment, the adjuvant is selected from, but is not limited to, an oil-in-water adjuvant, a polymer and water adjuvant, a water-in-oil adjuvant, an aluminum hydroxide adjuvant, a vitamin E adjuvant and combinations thereof. In another embodiment, the composition of the present invention further includes a pharmaceutically acceptable carrier.

The present also provides a method of immunizing an animal against C. jejuni, including a hypervirulent C. jejuni strain, the method including administering to the animal a composition of the present invention, as described above. This composition for administration includes the hypervirulent C. jejuni MOMP peptide, wherein polypeptide includes is SEQ ID NO: 1.

In one embodiment, the composition for administration includes a bacterium comprising and/or expressing the MOMP polypeptide. In another embodiment, the composition for administration includes an isolated, recombinant MOMP polypeptide.

In one embodiment of the method of the present invention, the composition can be administered intramuscularly, intradermally, transdermally, subcutaneously, intranasally, or orally, or by other routes known to those of skill in the art. In another embodiment, the composition is administered in a single dose. In yet another embodiment, the composition is administered as two or more doses.

In a further embodiment, the composition is administered to animals having maternally-derived antibodies against C. jejuni.

In one embodiment, the composition is administered to animals at 3 weeks of age or older, and prior to or during pregnancy.

The present invention further provides a kit. This kit includes a bottle comprising a vaccine composition according to the present invention for protecting animals against a highly virulent C. jejuni strain. This vaccine composition includes a C. jejuni MOMP polypeptide, of SEQ ID NO: 1.

In one embodiment of the kit, the vaccine composition is in the form of a bacterium comprising and/or expressing the MOMP polypeptide. In another embodiment of the kit, the vaccine composition is in the form of an isolated, recombinant MOMP polypeptide.

In one embodiment of the kit, the vaccine composition in the bottle is provided as a ready-to-use liquid composition. In another embodiment of the kit, the vaccine composition in the bottle is provided in a lyophilized form. In a further embodiment, the kit can include a diluent. In yet another embodiment, the kit can further include an instruction manual which contains the information for administration of the vaccine composition.

In addition, the invention includes method for diagnosing the existence of a C jejuni hypervirulent strain in an animal by detecting the presence of the MOMP protein of the invention. The presence of this protein may be determined by any of a number of tests known and understood by those of skill in the art. For example, suitable assays for use with the compositions and methods of the invention include, but are not limited to, antibody-based assays, nucleic acid-based assays, nucleic acid-based differential assays, such as fluorescent microsphere immunoassay, and real-time RT-PCR. Also included are diagnostic kits comprising these elements for diagnosis and detection of the MOMP of the invention to determine presence of the hypervirulent C. jejuni strain in a test subject.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Population genetics analysis and virulence properties of C. jejuni abortion isolates. (A) Maximum-likelihood phylogenetic tree based on whole genome SNP differences of 114 C. jejuni isolates and rooted to Campylobacter coli. Taxon names of clone SA isolates are not shown, but their branches are highlighted in red; and isolates from nonsheep sources are indicated by filled box of red (bovine abortion), purple (caprine abortion), orange (chicken meat), and green (human gastroenteritis). Nonclone SA references are indicated by blue. (Scale bar, number of nucleotide substitutions per site.) Root and strain 269.97 lengths are not to scale. (B) Regression analysis between isolation dates (x axis) and root-to-tip distance (y axis). The analysis was conducted based on 73 clone SA isolates. The point where the line intersects with the x axis gives the inferred date when the most recent common ancestor of C. jejuni clone SA emerged. The lone outlier CA12 is indicated by an arrow. (C) Virulence assessment of clone SA isolates (IA3902 and D7324) and nonclone SA isolates (VDL2401 and NCTC11168) in a pregnant guinea pig model. Survival curves (nonaborted pregnant/total pregnant) were compared by the log-rank (Mantel-Cox) test. Asterisk indicates a statistically significant difference from the NCTC11168-inoculated group (P<0.05).

FIG. 2. Identification of porA mutations responsible for the hypervirulence of C. jejuni clone SA. (A) Schematic depiction of the directed genome evolution strategy applied in this study. (B) Gain-of-function screening (positive selection) of NCTC11168 transformants that acquired the ability to cause abortion in pregnant guinea pigs. Three groups of animals were orally challenged with IA3902, NCTC11168, and NCTC11168 transformants, respectively. (C) Defining the role of porA in virulence by allele exchanges between IA3902 and NCTC11168. Pregnant guinea pig groups were challenged with NCTC11168, IA3902, NCTC11168 with IA3902 porA, and IA3902 with NCTC11168 porA, respectively. (D) Identification of the specific SNPs of porA responsible for virulence. Groups of pregnant guinea pigs were challenged with NCTC11168, NCTC11168 with loop 1 of IA3902 porA (loop 1); NCTC11168 with loop 3 of IA3902 porA (loop 3), NCTC11168 with loop 4 of IA3902 porA (loop 4), NCTC11168 with loops 1, 3, and 4 of IA3902 porA (loop 134), and NCTC11168 with the entire IA3902 porA (IA3902 porA), respectively. The predicted external loops of the porA-encoded protein are shown in FIG. 3C. In B-D, the asterisk indicates a statistically significant difference from the NCTC11168-inoculated group (P<0.05).

FIG. 3. Identification of small mutations (SNPs/indels) among the NCTC11168 transformants by Illumina sequencing 12 independent colonies and a pooled sample of colonies obtained from uterus and placenta of three aborted guinea pigs. (A) Heat map of 279 SNPs and 15 indels obtained from 12 independent colonies. The NCTC11168 recipient strain was resequenced in this study as a control. Each row represents a small mutation (SNP or indel) and colors indicate absence/presence (red/green) of the same mutation in a given sample (column labels). Mutations in 12 loci are unanimously present in all 12 colonies and the loci are *(mreB, cheA, cj0431, cj0455c, and cj0807), **(cj0046, cj1470c, cj0184c, and intergenic sites 1190424 and 1338383), cj1257c, and porA. (B) Plot of small mutations from the pooled sample (982 SNPs and 24 indels) using NCTC11168 as reference (x axis) and IA3902 (y axis). Mutational changes were collinear on the x-y axis with size of the dots proportional to allele frequency (red, high frequency; pink, intermediate frequency; blue, <1%), alleles with >99% frequency are indicated by arrows, and the related loci are shown. (C) Predicted secondary structures of MOMP (encoded by porA) of C. jejuni IA3902 (SEQ ID NO: 3) by PRED-TMBB and visualized by TMRPres2D. All of the amino acid level differences relative to C. jejuni NCTC11168 are highlighted by red circles.

FIG. 4. Evolutionary analysis of porA. (A) Working model for clone SA evolution. Clone SA (red) has undergone clonal expansion recently in clonal complex (CC) 21, driven by a selective advantage conferred by porA. Ongoing recombination exerts both loss of highly abortifacient potential in clone SA (black star) and gain of the same in nonclone SA strains (VDL902 and VDL35490). (B and C) Genome-wide sliding window analysis (window size, 1 kb; step size, 500 bp). (B) Plot of Fu's Fs statistic (y axis) and the genomic coordinate of the center of the window (x axis). (C) Plot of the number of haplotypes (y axis) versus the number of segregating sites (x axis, clipped at 100). Windows containing porA are indicated with larger red dots (B and C). (D) Mutation map of the porA locus. Each allele (haplotype) of a 2-kb window surrounding porA is depicted (red line). The number of strains and the number of mutations differentiating that allele from the consensus is indicated (Right). Black vertical lines indicate the location of a mutation. Allele count per mutation per position is shown (Bottom). Those with one allele carrying the mutation are singletons. The genomic organization of porA loop 4 region (dark blue) and neighboring genes are depicted (Bottom).

FIG. 5. Geographical and temporal distribution of clone SA (A) and non-clone SA (B) C. jejuni isolates sequenced in this study.

FIG. 6. Graphical representation of recombination across Campylobacter jejuni genome sequences as determined by BRATNextGen analysis. Clone SA isolates have significantly less recombination, suggesting that they are a clonal population. On the left side of zero, the PSA tree is shown. On the right side of zero, the segments detected in each sample are shown as colored stripes. The same color at the same column means that the segments in the respective samples are from the same origin. A continuous stretch shaded with a single color represents a single recombinogenic segment.

FIG. 7. Circular representation of the genomic comparison between C. jejuni NCTC11168 and C. jejuni VDL2401 (from ovine abortion case) as an example to show the closely related genomes. The outer circle shows the genome scale of NCTC11168. The second circle denotes genomic alignment of VDL2401 contigs to NCTC11168 complete genome. The third circle displays the sole 15 mutations detected between VDL2401 and NCTC11168.

FIG. 8. Gene presence-absence analyses did not capture definitive genetic basis of the hypervirulence in abortion induction. A binary heat map of gene presence (green) and absence (red) for every orthologous gene across 114 samples (columns) was plotted using protein coding genes of IA3902 as reference (rows; n=1587). The profile differentiated 2 distinct clades; clone SA and non-clone SA, however, no genes were specifically present and conserved among clone SA isolates. porA was ubiquitously present in all samples.

FIG. 9. Construction of a saturated pool of transformants of NCTC11168. After four times of transformation by C. jejuni IA3902 genomic DNA, the proportion of tetracycline resistant colonies plateaued (approximately 1/10,000).

FIG. 10. Bacterial loads in uteroplacental unit of aborted guinea pigs. Pregnant guinea pigs were orally challenged with ˜10⁹ CFU Campylobacter. Necropsies were performed at the time of abortion or at 20/21 days after inoculation in guinea pigs that did not abort. (A) Campylobacter CFUs recovered from aborted guinea pigs of Group NCTC11168 transformants are shown; (B) Campylobacter CFUs recovered from aborted guinea pigs of Group IA3902 with NCTC11168 porA and Group NCTC11168 with IA3902 porA are shown. Non-aborted animals of all groups were always negative for Campylobacter in the uteroplacental tissues as determined at necropsy. Mann-Whitney U test was performed to compare the CFUs between groups for statistical significance (*p<0.05).

FIG. 11. PFGE profiles of the isolates from placenta and uterus of aborted guinea pigs inoculated with NCTC11168 transformants. PFGE was run as described previously.

FIG. 12. Mapping pipeline for small mutations enables identification of true SNP/Indels between NCTC11168 and IA3902. Left: Coverage plot of all true SNPs (green dots) and Indels (red) along the genome (NCTC11168-X axis and IA3902-Y axis); Right: the workflow for SNP/Indel calling pipeline.

FIG. 13. Schematic depiction of the porA gene replacement strategy between IA3902 and NCTC11168. Kanamycin resistance cassette (KmR) was inserted in the intergenic region between porA and dnaJ. Primers used are indicated by arrows.

FIG. 14A-D. Phylogenetic analysis of porA sequences and recombination analysis of porA in CA12. (A-C) Maximum likelihood tree of porA sequences from all the strains in this study. Colors indicate groups of strains as indicated in the legend. Clone SA isolates (red and green labels) are determined by whole genome phylogeny as described in the main text. CA12 belongs to clone SA, but its porA is closely related to a non-clone SA strain ICDCCJ07001 (indicated by a red arrow). (D) Recombination analysis of porA of strain CA12. porA sequences from strain CA12, IA3902 and ICDCCJ07001 were aligned. Mutation distribution of CA12 porA is close to ICDCCJ07001, however CA12's gene allelic profile is identical with IA3902 for most genes chosen from other random regions across the entire genome (4 representative regions are shown in the figure). This observation indicates that recombination changes the allelic profile of CA12's porA and makes it close to porA of ICDCC J07001 (non-Clone SA).

FIG. 15. Evolutionary analysis of genome sequences. (A) Venn diagram depicting the data subsets used in the analyses. The white box represents all strains (114 genomes total). Different colored areas represent other subsets of the data. Each subset has a group label (i-iii) by which it is referred to (i.e. All isolates: i-114 isolates; All Clone SA: ii-73 isolates from both abortion and other sources; Clone SA abortion: iii-59 isolates; and Clone SA-abortion excluding CA12: iii w/o CA12-58 isolates). Strain CA12 is the only strain in set (iii) that appears to have a recombination at the porA locus. (B) Summary of recombination and evolutionary analyses performed on the data sets depicted in (a). + indicates that porA was identified to have recombination or gave a statistically significant signal (see methods for details on significance cutoffs). (C) Genome-wide sliding window analysis of the number of singletons (mutations present in only a single allele of data set (iii) excluding CA12) per windows. Data is shown for a window size of 1 kb with a step size of 0.5 kb. Windows that overlap porA are shown as larger red circles. (D) Relationship of Fu's Fs statistic to the number of haplotypes and the number of segregating sites per window. Windows are the same as in (C). Fu's Fs is plotted on the y-axis, haplotypes per window on the x-axis, and the number of segregating sites is indicated by the size of the data points. Horizontal line indicates the cutoff for significance (see methods for details). Red data points indicate windows that overlap porA.

FIG. 16. SDS-PAGE and Immunoblotting showing the reactivity of the rabbit anti-loop 4 antibody with whole cell lysate of C. jejuni IA3902 and W7. The antibody specifically recognized MOMP in both strains, but the reactivity to IA3902 is stronger than to W7.

FIG. 17. Immunoblotting analysis of antibody responses in immunized guinea pig sera. (A) Dot blotting of guinea pig sera against the recombined loop 4 peptide. (B) Western blotting of guinea pig sera against whole cell lysate of C. jejuni IA3902. NC is negative control (pre-immunized guinea pig serum), while PC is the rabbit anti-loop 4 antibody. All the other samples are individual sera from immunized guinea pigs. The position of MOMP in panel B is indicated by an arrow.

FIG. 18. Immunization of pregnant guinea pigs with a recombinant loop 4 peptide vaccine against challenge with C. jejuni IA3902. The sham group (n=10) was subcutaneously injected with the aluminum hydroxide gel adjuvant only, while the vaccinated group (n=14) was subcutaneously injected with the recombinant loop 4 vaccine. Survival rate indicate the proportion of non-aborted animals. The challenge (post inoculation) last 21 days and is still ongoing. The data presented here is what has been obtained so far.

### DETAILED DESCRIPTION OF THE INVENTION

As used in the specification and claims, the singular form “a”, “an” and “the” include plural references unless the context clearly dictates otherwise. For example, the term “a protein antigen” includes a plurality of protein antigens, including mixtures thereof.

As used herein, the term “comprising” is intended to mean that the compositions and methods include the recited elements, but do not exclude other elements.

As used herein, the terms “hypervirulent C. jejuni strain”, “hyper virulent C. jejuni”, “C. jejuni mutant”, “novel mutant C. jejuni”, “mutant C. jejuni”, and the like refer to a highly virulent C. jejuni strain that induced abortion and includes a specific peptide in the major outer membrane protein (MOMP) for its hyper-virulence in inducing abortion. The peptide sequence (AEEQGADLLGKSTISTTQKAAPFQADSLGNL) (SEQ ID NO: 1) is located in the predicted external loop 4 of MOMP encoded by porA. The loop 4 sequence is critical for virulence.

As used herein, the term “a C. jejuni hypervirulence peptide (or polypeptide)” is intended to include a bacterium comprising and/or expressing the C. jejuni hypervirulence polypeptide, such that the MOMP peptide of SEQ ID NO:1 is a component of the bacterium itself (e.g., membrane protein of the bacterium). The bacterium can be C. jejuni, but should not be construed to be limited to such, and can include other bacteria such as Escherichia coli. This term is also intended to include an isolated, recombinant C. jejuni hypervirulence polypeptide.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). The term “antigen” can include a whole bacterium, a polypeptide, or a fragment thereof.

As used herein, the term “vaccine composition” includes at least one antigen or immunogen in a pharmaceutically acceptable vehicle useful for inducing an immune response in a host. Vaccine compositions can be administered in dosages, and by techniques well known to those skilled in the medical or veterinary arts, taking into consideration factors such as the age, sex, pregnancy, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences” (1990), may be consulted to prepare suitable preparations, without undue experimentation.

As defined herein, an “immunogenic or immunological composition”, refers to a composition of matter that comprises at least one antigen which elicits an immunological response in the host of a cellular and/or antibody-mediated immune response to the composition or vaccine of interest.

The term “immune response” as used herein refers to a response elicited in an animal. An immune response may refer to cellular immunity (CMI), humoral immunity, or may involve both. The present invention also contemplates a response limited to a part of the immune system. Usually, an “immunological response” includes, but is not limited to, one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response, such that resistance to new infection will be enhanced, and/or the clinical severity of the disease reduced. Such protection will be demonstrated by either a reduction or lack of symptoms normally displayed by an infected host, a quicker recovery time, and/or a lowered bacterial titer in the infected host.

As used herein, the term “immunogenicity” means capable of producing an immune response in a host animal against an antigen or antigens. This immune response forms the basis of the protective immunity elicited by a vaccine against a specific infectious organism.

An “adjuvant” as used herein means a composition comprised of one or more substances that enhances the immune response to an antigen(s). The mechanism of how an adjuvant operates is not entirely known. Some adjuvants are believed to enhance the immune response by slowly releasing the antigen, while other adjuvants are strongly immunogenic in their own right, and are believed to function synergistically.

As used herein, the term “multivalent” means a vaccine containing more than one antigen, whether from the same microbiological species (e.g., different isolates of C. jejuni), from different species (e.g., isolates from Campylobacter), or a vaccine containing a combination of antigens from different genera (for example, a vaccine comprising antigens from C. jejuni, Salmonella, Escherichia coli, Haemophilus somnus and Clostridium).

As used herein, the term “virulent” means an isolate that retains its ability to be infectious in an animal host and is capable of causing disease in the host animal.

“Inactivated vaccine” means a vaccine composition containing an infectious organism or pathogen that is no longer capable of replication or growth. The pathogen may be bacterial, viral, protozoal or fungal in origin. Inactivation may be accomplished by a variety of methods, including freeze-thawing, chemical treatment (for example, treatment with β-propiolactone (BPL) or formalin), sonication, radiation, heat, or any other conventional means sufficient to prevent replication or growth of the organism, while maintaining its immunogenicity.

The term “variant” as used herein refers to a polypeptide or a nucleic acid sequence encoding a polypeptide, that has one or more conservative amino acid variations or other minor modifications such that the corresponding polypeptide has substantially equivalent function when compared to the wild-type polypeptide. The term “variant” can also refer to a microorganism comprising a polypeptide or nucleic acid sequence having said variations or modifications as well.

“Conservative variation” denotes the replacement of an amino acid residue by another biologically similar residue, or the replacement of a nucleotide in a nucleic acid sequence such that the encoded amino acid residue does not change, or is another biologically similar residue. Examples of conservative variations include the substitution of one hydrophobic residue, such as isoleucine, valine, leucine or methionine, for another hydrophobic residue, or the substitution of one polar residue with another, such as the substitution of arginine for lysine, glutamic acid for aspartic acid, or glutamine for asparagine, and the like. The term “conservative variation” also includes a substituted amino acid in place of a parent amino acid, provided that antibodies raised to the substituted polypeptide also immunoreact with the parent (unsubstituted) polypeptide.

As used herein, the terms “pharmaceutically acceptable carrier” and “pharmaceutically acceptable vehicle” are interchangeable, and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, coloring additives, and the like.

As used herein, a genetically modified bacterium is “attenuated” if it is less virulent than its unmodified parental strain. A strain is “less virulent” if it shows a statistically significant decrease in one or more parameters determining disease severity. Such parameters may include level of bacteremia, fever, abortion, severity of reproductive symptoms, severity of diarrhea or number or severity of pathological lesions, etc.

The present invention provides a vaccine composition for protecting animals against C. jejuni, including the hypervirulent clone (SA) of C. jejuni, the composition including a C. jejuni MOMP polypeptide, wherein the MOMP polypeptide comprises Glutamic Acid (E) at position 3, Lysine (K) at position 11, Threonine (T) at position 13, Glutamine (Q) at position 18, Lysine (K) at position 19, Alanine (A) at position 20, Glutamine (Q) at position 24, Alanine (A) at position 25, and Leucine (L) at position 28 according to the numbering of SEQ ID NO: 1.

In one embodiment, the C. jejuni MOMP polypeptide is SEQ ID NO: 1 or its conservatively modified variants or any other variants with immunogenic activity.

In one embodiment, the C. jejuni MOMP polypeptide is represented by the amino acid sequence of SEQ ID NO: 1 or a fragment thereof. However, the present invention is not limited to this embodiment. For example, in other embodiments, the C. jejuni MOMP polypeptide can be selected from, but is not limited to conservatively modified variants, fragments and derivatives thereof capable of eliciting an immune response.

In one embodiment, the vaccine compositions of the present invention include at least one additional antigen. In one embodiment, the at least one additional antigen is protective against a disease in animals caused by a microorganism.

In some embodiments, the at least one additional antigen component is protective against a disease in animals caused by bacteria, viruses, or protozoans that are known to infect animals. Examples of such microorganisms include, but are not limited to, the following: Campylobacter jejuni, Campylobacter fetus, Haemophilus parasuis, Pasteurella multocida, Streptococcum suis, Staphylococcus hyicus, Actinobacilllus pleuropneumoniae, Bordetella bronchiseptica, Salmonella choleraesuis, Salmonella enteritidis, Erysipelothrix rhusiopathiae, and Escherichia coli, or combinations thereof.

In one embodiment, the at least one additional antigen is a non-MOMP antigen in Campylobacter jejuni. In another embodiment, the at least one additional antigen is Campylobacter fetus. It is also anticipated that the at least one additional antigen can be a different isolate of C. jejuni, such as a classical C. jejuni strain, or other C. jejuni genotypes.

In one embodiment, the composition is in the form of an inactivated, hypervirulent C. jejuni whole bacterium that comprises and/or expresses the MOMP polypeptide.

In one embodiment, the MOMP gene (porA) of the hypervirulent C. jejuni whole bacterium corresponds to sequence disclosed herein and in Genbank associated with C. jejuni. In a further embodiment, the amino acid sequence of the hypervirulent C. jejuni MOMP polypeptide which is expressed by C. jejuni corresponds to SEQ ID NO: 1 or a fragment thereof. However, the present invention is not limited to these embodiments.

In another embodiment, the composition is in the form of an inactivated recombinant strain, wherein the recombinant strain comprises an inactivated recombinant bacterium that comprises and/or expresses the MOMP polypeptide (chimeric C. jejuni virus).

In one embodiment, the MOMP gene of the chimeric C. jejuni virus corresponds to SEQ ID NO: 2. In a further embodiment, the amino acid sequence of the MOMP polypeptide which is expressed by the recombinant C. jejuni strain or another bacterium (such as Escherichia coli) corresponds to SEQ ID NO: 1 or a fragment thereof. However, the present invention is not limited to these embodiments.

In yet another embodiment, the composition is in the form of an isolated, recombinant C. jejuni MOMP polypeptide or multiple copies of the same peptide. In one embodiment, the isolated, recombinant C. jejuni MOMP polypeptide is expressed from a vector, such as a plasmid. Alternatively, other known expression vectors can be used, such as including, but not limited to, Escherichia coli. In one embodiment, the vector can be a live or inactivated vector.

In a further embodiment, the recombinantly-expressed C. jejuni MOMP polypeptide corresponds to SEQ ID NO: 1 or a fragment thereof. Alternatively, in some embodiments, the recombinantly-expressed C. jejuni MOMP polypeptide can be selected from any fragments or derivative thereof capable of eliciting an immune response.

In some forms, immunogenic portions of the C. jejuni MOMP protein are used as the antigenic component in the composition. For example, truncated and/or substituted forms or fragments of the C. jejuni MOMP protein may be employed in the compositions of the present invention.

It is understood by those of skill in the art that variants of the hypervirulent C. jejuni MOMP polypeptides can be employed in the compositions of the present invention, provided they still retain the antigenic characteristics that render it useful in the vaccine compositions of this invention. Preferably, C. jejuni hypervirulent variants have at least 80%, preferably at least 85%, more preferably at least 90%, even more preferably at least 95% sequence identify with the full-length genomic sequence of the C. jejuni isolate termed C. jejuni_IA3902. The antigenic characteristics of an immunological composition can be, for example, estimated by the challenge experiment as provided in the Examples. Moreover, the antigenic characteristic of a modified hypervirulent C. jejuni MOMP antigen is still retained when the modified antigen confers at least 70%, preferably 80%, more preferably 90% of the protective immunity as compared to the wild-type C. jejuni MOMP protein having SEQ ID NO: 1.

The hypervirulent C. jejuni MOMP antigen component is provided in the immunogenic/vaccine composition at an antigen inclusion level effective for inducing the desired immune response, namely reducing the incidence of or lessening the severity of clinical signs resulting from infection with a highly virulent C. jejuni strain, an example of which is the abortifacient clone termed C. jejuni_clone SA_herein. In some embodiments, the composition also provides heterologous protection against classical C. jejuni strains.

In one embodiment, a vaccine composition according to the present invention is in the form of an inactivated recombinant bacterium that comprises and/or expresses the C. jejuni MOMP polypeptide (recombinant strain). This recombinant bacterium is included in the compositions of the invention at a level of at least 1.0≤RP≤5.0, wherein RP is the Relative Potency unit determined by ELISA antigen quantification (in vitro potency test) compared to a reference vaccine. In another embodiment, a recombinant strain is included in the composition of the invention at a final concentration of about 0.5% to about 5% of 20-times (20×) concentrated bulk C. jejuni MOMP antigen.

In another embodiment, a vaccine composition according to the present invention is in the form of an inactivated, hypervirulent C. jejuni whole bacterium that comprises and/or expresses the C. jejuni MOMP polypeptide. This bacterium is included in the compositions of the invention at a level of at least 1.0≤RP≤5.0, wherein RP is the Relative Potency unit determined by ELISA antigen quantification (in vitro potency test) compared to a reference vaccine. In another embodiment, an inactivated hypervirulent C. jejuni whole bacterium is included in the composition of the invention at a final concentration of about 0.5% to about 5% of 20-times (20×) concentrated bulk C. jejuni MOMP antigen.

In yet another embodiment, a vaccine composition according to the present invention is in the form of an isolated, recombinant C. jejuni MOMP polypeptide. The C. jejuni MOMP recombinant protein can be included in the compositions of the invention at a level of at least 0.2 μg antigen/ml of the final immunogenic/vaccine composition (μg/ml). In a further embodiment, the recombinant C. jejuni MOMP polypeptide inclusion level is from about 0.2 to about 400 μg/ml. In yet another embodiment, the recombinant C. jejuni MOMP polypeptide inclusion level is from about 0.3 to about 200 μg/ml. In a still further embodiment, the recombinant C. jejuni hypervirulent MOMP polypeptide inclusion level is from about 0.35 to about 100 μg/ml. In still another embodiment, the recombinant C. jejuni MOMP polypeptide inclusion level is from about 0.4 to about 50 μg/ml.

In one embodiment, a vaccine composition of the present invention includes the combination of the C. jejuni MOMP polypeptide, and at least one additional soluble antigen (e.g., two or more).

In one embodiment, a C. jejuni MOMP vaccine is provided as a single-dose, 1-bottle vaccine. In another embodiment, a C. jejuni MOMP vaccine is provided as a multi-dose vaccine. In some embodiments, additional antigens can be added to either the single or the multi-dose vaccine.

Vaccines and/or immunogenic compositions of the present invention can be formulated following accepted convention to include pharmaceutically acceptable carriers for animals, including humans (if applicable), such as standard buffers, stabilizers, diluents, preservatives, and/or solubilizers, and can also be formulated to facilitate sustained release. Diluents include water, saline, dextrose, ethanol, glycerol, and the like. Additives for isotonicity include sodium chloride, dextrose, mannitol, sorbitol, and lactose, among others. Stabilizers include albumin, among others. Other suitable vaccine vehicles and additives, including those that are particularly useful in formulating modified live vaccines, are known or will be apparent to those skilled in the art. See, e.g., Remington's Pharmaceutical Science, 18th ed., 1990, Mack Publishing, which is incorporated herein by reference.

Vaccines or immunogenic compositions of the present invention can further comprise one or more additional immunomodulatory components such as, e.g., an adjuvant or cytokine, among others. Types of suitable adjuvants for use in the compositions of the present invention include the following: an oil-in-water adjuvant, a polymer and water adjuvant, a water-in-oil adjuvant, an aluminum hydroxide adjuvant, a vitamin E adjuvant and combinations thereof Some specific examples of adjuvants include, but are not limited to, complete Freund's adjuvant, incomplete Freund's adjuvant, Corynebacterium parvum, Bacillus Calmette Guerin, aluminum hydroxide gel, glucan, dextran sulfate, iron oxide, sodium alginate, Bacto-Adjuvant, certain synthetic polymers such as poly amino acids and co-polymers of amino acids, Block copolymer (CytRx, Atlanta, Ga.), QS-21 (Cambridge Biotech Inc., Cambridge Mass.), SAF-M (Chiron, Emeryville Calif.), AMPHIGEN® adjuvant, saponin, Quil A or other saponin fraction, monophosphoryl lipid A, and Avridine lipid-amine adjuvant (N,N-dioctadecyl-N′,N′-bis(2-hydroxyethyl)-propanediamine), “REGRESSIN” (Vetrepharm, Athens, Ga.), paraffin oil, RIBI adjuvant system (Ribi Inc., Hamilton, Mont.), muramyl dipeptide and the like.

Non-limiting examples of oil-in-water emulsions useful in the vaccine of the invention include modified SEAM62 and SEAM ½ formulations. Modified SEAM62 is an oil-in-water emulsion containing 5% (v/v) squalene (Sigma), 1% (v/v) SPAN® 85 detergent (ICI Surfactants), 0.7% (v/v) TWEEN® 80 detergent (ICI Surfactants), 2.5% (v/v) ethanol, 200 μg/ml Quil A, 100 μg/ml cholesterol, and 0.5% (v/v) lecithin. Modified SEAM ½ is an oil-in-water emulsion comprising 5% (v/v) squalene, 1% (v/v) SPAN® 85 detergent, 0.7% (v/v) Tween 80 detergent, 2.5% (v/v) ethanol, 100 μg/ml Quil A, and 50 μg/ml cholesterol. In a preferred embodiment the adjuvant or other additional component is one with which the bacterium or antigenic protein segment is not associated or present with in its natural state.

Another example of an adjuvant useful in the compositions of the invention is SP-oil. As used in the specification and claims, the term “SP oil” designates an oil emulsion comprising a polyoxyethylene-polyoxypropylene block copolymer, squalane, polyoxyethylene sorbitan monooleate and a buffered salt solution. Polyoxyethylene-polyoxypropylene block copolymers are surfactants that aid in suspending solid and liquid components. These surfactants are commercially available as polymers under the trade name Pluronic®. The preferred surfactant is poloxamer 401 which is commercially available under the trade name Pluronic® L-121. In general, the SP oil emulsion is an immunostimulating adjuvant mixture which will comprise about 1 to 3% vol/vol of block copolymer, about 2 to 6% vol/vol of squalane, more particularly about 3 to 6% of squalane, and about 0.1 to 0.5% vol/vol of polyoxyethylene sorbitan monooleate, with the remainder being a buffered salt solution. In one embodiment, the SP-oil emulsion is present in the final composition in v/v amounts of about 1% to 25%, preferably about 2% to 15%, more preferably about 5% to 12% v/v.

Yet another example of a suitable adjuvant for use in the compositions of the invention is AMPHIGEN™ adjuvant which consists of de-oiled lecithin dissolved in an oil, usually light liquid paraffin.

Other examples of adjuvants useful in the compositions of the invention are the following proprietary adjuvants: Microsol Diluvac Forte® duel emulsion adjuvant system, Emunade adjuvant, and Xsolve adjuvant. Both the Emunade and Xsolve adjuvants are emulsions of light mineral oil in water, but Emunade also contains alhydrogel, and d,1-α-tocopheryl acetate is part of the XSolve adjuvant. A still further example of a suitable adjuvant for use in the compositions of the invention is ImpranFLEX™ adjuvant (a water-in-oil adjuvant). A still further example of a suitable adjuvant is a Carbomer (Carbopol®) based adjuvant. Preferred Carbopol® adjuvants include Carbopol® 934 polymer and Carbopol® 941 polymer.

In one embodiment, the adjuvant or adjuvant mixture is added in an amount of about 100 μg to about 10 mg per dose. In another embodiment, the adjuvant/adjuvant mixture is added in an amount of about 200 μg to about 5 mg per dose. In yet another embodiment, the adjuvant/adjuvant mixture is added in an amount of about 300 μg to about 1 mg/dose.

The adjuvant or adjuvant mixture is typically present in the vaccine or immunogenic composition of the invention in v/v amounts of about 1% to 25%, preferably about 2% to 15%, more preferably about 5% to 12% v/v.

Other “immunomodulators” that can be included in the compositions of the invention include, e.g., one or more interleukins, interferons, or other known cytokines In one embodiment, the adjuvant may be a cyclodextrin derivative or a polyanionic polymer, such as those described in U.S. Pat. Nos. 6,165,995 and 6,610,310, respectively.

The present invention also provides a method of immunizing an animal against a hypervirulent C. jejuni strain, the method including administering to the animal a composition according to the present invention, as described above. This composition for administration includes a C. jejuni MOMP polypeptide, wherein the MOMP polypeptide includes SEQ ID NO: 1, fragments, derivatives or conservatively modified variants thereof.

In one embodiment, the composition for administration includes a bacterium comprising and/or expressing the C. jejuni MOMP polypeptide. In another embodiment, the composition for administration includes an isolated, recombinant C. jejuni MOMP polypeptide.

In one embodiment of the method of the present invention, the composition is administered intramuscularly, intradermally, transdermally, subcutaneously, or orally. In another embodiment, the composition is administered in a single dose. In yet another embodiment, the composition is administered as two or more doses.

In a further embodiment, the composition is administered to animals prior to or during pregnancy.

In one embodiment, the composition is administered to animals at 3 weeks of age or older.

Vaccine and/or immunogenic compositions according to the present invention can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions according to the present invention can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration), such as sterile suspensions or emulsions. Vaccine compositions according to the present invention may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

The present invention further provides a kit. This kit includes a bottle containing a vaccine composition according to the present invention for protecting animals against a hypervirulent strain of C. jejuni. This vaccine composition includes a C. jejuni MOMP polypeptide, wherein the MOMP polypeptide of SEQ ID NO: 1, or an immunogenic fragment thereof.

In one embodiment of the kit, the vaccine composition is in the form of a bacterium comprising and/or expressing the C. jejuni MOMP polypeptide. In another embodiment of the kit, the vaccine composition is in the form of an isolated, recombinant C. jejuni MOMP polypeptide.

In one embodiment of the kit of the present invention, the vaccine composition in the bottle is provided as a ready-to-use liquid composition. In another embodiment of the kit, the vaccine composition in the bottle is provided in a lyophilized form. In a further embodiment, the kit can include a diluent. In yet another embodiment, the kit can further include an instruction manual which contains the information for administration of the vaccine composition.

A further aspect of the present invention provides methods of producing a vaccine composition which is in the form of an inactivated, hypervirulent C. jejuni whole bacterium that expresses the C. jejuni MOMP polypeptide. In one embodiment, the final composition is prepared by combining the inactivated hypervirulent C. jejuni strain with a suitable adjuvant and/or other pharmaceutically acceptable carrier.

Yet another aspect of the present invention provides methods of producing a recombinant C. jejuni MOMP protein, i) by permitting transformation of E. coli with a recombinant vector (such as a plasmid) containing the hypervirulent C. jejuni MOMP DNA coding sequences, wherein MOMP peptide is expressed from the recombinant vector, and ii) thereafter recovering the MOMP peptide expressed in the E. coli. Typically, high amounts of C. jejuni MOMP peptide can be recovered from E. coli. High amounts of C. jejuni hypervirulent MOMP means more than about 20 μg/mL supernate, preferably more than about 25 μg/mL, even more preferred more than about 30 μg/mL, even more preferred more than about 40 μg/mL, even more preferred more than about 50 μg/mL, even more preferred more than about 60 μg/mL, even more preferred more than about 80 μg/mL, even more preferred more than about 100 μg/mL, even more preferred than about 150 μg/mL, most preferred than about 190 μg/mL.

Preferred cell cultures have a cell count between about 0.3-2.0×106 cells/mL, more preferably from about 0.35-1.9×106 cells/mL, still more preferably from about 0.4-1.8×106 cells/mL, even more preferably from about 0.45-1.7×106 cells/mL, and most preferably from about 0.5-1.5×106 cells/mL. Preferred cells are determinable by those of skill in the art. Preferred cells are those susceptible for infection with an appropriate recombinant viral vector, containing a C. jejuni hypervirulent MOMP DNA and expressing the C. jejuni hypervirulent MOMP protein. Preferably the cells are insect cells, and more preferably, they include the insect cells sold under the trademark Sf+ insect cells (Protein Sciences Corporation, Meriden, Conn.).

Yet another aspect of the present invention provides methods of producing recombinant C. jejuni MOMP peptide, i) by concatenating four copies of the DNA encoding loop 4 (SEQ ID NO: 1) on a plasmid and cloning into host E. coli, wherein MOMP peptide is expressed by the recombinant plasmid vector, and ii) thereafter recovering the MOMP peptide in the host E. coli cells. Typically, high amounts of the C. jejuni MOMP peptide can be recovered in the supernantant of sonicated E. coli cells. High amounts of C. jejuni MOMP means more than about 5 μg/mL supernate, preferably more than about 10 μg/mL, even more preferred more than about 30 μg/mL, even more preferred more than about 40 μg/mL, even more preferred more than about 50 μg/mL, even more preferred more than about 60 μg/mL, even more preferred more than about 80 μg/mL, even more preferred more than about 100 μg/mL, even more preferred than about 150 μg/mL, most preferred than about 190 μg/mL.

Preferred E. coli cultures have a cell density between about 2.5-4×10⁸ cells/mL, more preferably from about 4-5×10⁸ cells/mL, still more preferably from about 5-6×10⁸ cells/mL, even more preferably from about 6-7×10⁸ cells/mL, and most preferably from about 7-10×10⁸ cells/mL. Preferred cells are determinable by those of skill in the art. Preferred cells are host E. coli with an appropriate recombinant plasmid vector, containing C. jejuni MOMP DNA and expressing the C. jejuni MOMP peptide.

Appropriate growth media will also be determinable by those of skill in the art with a preferred growth media such as LB (Luria-Bertani) (BD Difco™, Franklin Lakes, N.J. and the like. Preferred plasmid vectors include plasmids such as pQE-1 (QIAGEN Inc, Valencia, Calif.). Although the pQE-1 in E. coli JM109 expression system is preferred, it is understood by those of skill in the art that other expression systems will work for purposes of the present invention. When MOMP is produced by a pQE-1 expression system, then typically it does not include any signal sequence and MOMP will be expressed in the cytoplasm of E. coli host cells. The expressed protein product contains four copies of Loop4 of MOMP and 3 copies of the linker and the His N-terminal leader sequence

### (MKHHHHHHQLHAGAHAEEQGADLLGKSTISTTQKAAPFQADSLGNLGGSSGGA EEQGADLLGKSTISTTQKAAPFQADSLGNLGGSSGGAEEQGADLLGKSTISTTQKA APFQADSLGNLGGSSGGAEEQGADLLGKSTI STTQKAAPFQADSLGNL) (SEQ ID NO: 4).

The transformed E. coli cells by pQE-1 expression system are incubated at 37° C. over a period of 3 hours and the expression of the MOMP peptide is then induced by adding 1 mM isopropyl-D-thiogalactoside (IPTG) at 37° C. Preferred induction time is 4 hours, more preferably from about 4-5 hours., still more preferably from about 5-6 hours.

The recovery process preferably begins with the collection of E. coli cells from culture media via centrifugation. Preferred steps include centrifugation at speeds up to about 5,000×g to collect the E. coli cells, sonication to break down the E. coli cell and release the cytoplasmic content which contains the expressed recombinant C. jejuni MOMP polypeptide, and affinity purification using Ni-NTA technology. Those methods are detailed in Qiagen purification manual—The QIAexpressionist (QIAGEN Inc, Valencia, Calif.).

A further aspect of the present invention relates to a method for preparing a composition comprising C. jejuni MOMP peptide. This method includes the steps: i) cloning the C. jejuni MOMP recombinant structure into an expression plasmid; ii) transforming the plasmid containing the recombinant C. jejuni MOMP gene into E. coli; iii) inducing the transformed plasmid to express the C. jejuni MOMP recombinant protein from C. jejuni MOMP Loop4 DNA structure; iv) recovering and purifying the expressed C. jejuni MOMP protein from E. coli. Preferably, the recombinant plasmid is pQE-1, and the host is E. coli JM109. Preferred induction, separation and purification are those described above.

In another aspect of the present invention, a method for preparing a composition, preferably an antigenic composition, such as for example a vaccine, for invoking an immune response against a hypervirulent C. jejuni strain is provided. Generally, this method includes the steps of transforming a construct into E. coli, wherein the construct comprises i) recombinant DNA expressing MOMP of a hypervirulent C. jejuni strain; ii) transforming the plasmid into E. coli and growing in media; iii) inducing the plasmid to express the recombinant protein; iv) recovering the expressed MOMP protein from the supernatant; v) and preparing the composition by combining the recovered protein with a suitable adjuvant and/or other pharmaceutically acceptable carrier.

Antibodies

Also contemplated by the present invention are anti-C jejuni antibodies (e.g., monoclonal and polyclonal antibodies, single chain antibodies, chimeric antibodies, humanized, human, animal, and CDR-grafted antibodies, including compounds which include CDR sequences which specifically recognize a MOMP of the invention. The term “specific for” indicates that the variable regions of the antibodies of the invention recognize and bind a C jejuni MOMP exclusively (i.e., are able to distinguish a single C. jejuni polypeptide from related polypeptides despite sequence identity, homology, or similarity found in the family of polypeptides), and which are permitted (optionally) to interact with other proteins through interactions with sequences outside the variable region of the antibodies, and in particular, in the constant region of the Ab molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies that recognize and bind fragments of the C. jejuni polypeptides of the invention are also contemplated, provided that the antibodies are first and foremost specific for, as defined above, a C. jejuni polypeptide of the invention from which the fragment was derived.

For the purposes of clarity, “antibody” refers to an immunoglobulin molecule that can bind to a specific antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. Antibodies can exist in a variety of forms including, for example, as, Fv, Fab′, F(ab′) 2, as well as in single chains, and include synthetic polypeptides that contain all or part of one or more antibody single chain polypeptide sequences.

### Diagnostic Kits

The present invention also provides diagnostic kits. The kit can be valuable for identifying animals potentially infected with hypervirulent C. jejuni can be detected prior to the existence of clinical symptoms so that these animals can be treated or removed from places where they might infect other animals. The kits include reagents for analyzing a sample from an animal for the presence of a MOMP of the invention. In addition, the invention includes method for diagnosing the existence of a C jejuni hypervirulent strain in an animal by detecting the presence of the MOMP protein of the invention. The presence of this protein may be determined by any of a number of tests known and understood by those of skill in the art. For example, suitable assays for use with the compositions and methods of the invention include, but are not limited to, antibody-based assays, nucleic acid-based assays, nucleic acid-based differential assays, such as fluorescent microsphere immunoassay, and real-time RT-PCR.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### Example 1

### Introduction

Pathogens evolve relentlessly, often resulting in regional or global expansion of successful clones or strains. In many of these cases, pathogens causing outbreaks are hypothesized to have increased virulence, due to acquisition of new transmission, survival, or infection traits(1-7). Knowledge of the precipitating genetic and phenotypic change(s) responsible for virulence is necessary for guiding rational design of effective control measures. Whole genome sequencing has provided a powerful tool to identify such evolutionary genetic changes and has transformed the ways by which we understand bacterial virulence, pathogenesis, epidemiology, and evolution (8). However, the elucidation of exact mechanisms underlying the success of individual pathogenic clones remains difficult, especially for recently emergent and expanding clones with enhanced virulence. In these cases, we are often left only with correlated genetic markers and lack information on causative changes. This is especially true when the success of a pathogenic clone may only involve minor genetic changes, such as single nucleotide polymorphisms.

Campylobacter jejuni is a major foodborne pathogen and a leading cause of enteritis in humans, responsible for 400-500 million cases of diarrhea annually worldwide(9). In the U.S. alone, Campylobacter accounts for more than 800,000 cases of foodborne illnesses each year (10). As a zoonotic pathogen, C. jejuni is widely distributed in the gut microbiota of wild and domesticated animal species, such as cattle, sheep, and poultry(11, 12). Transmission of C. jejuni to humans is mainly via contaminated meat, milk, and water. Although C. jejuni is primarily a gut colonizer, some hypervirulent strains may be able to translocate across intestinal epithelium, producing bacteremia and systemic infections(13). In addition to causing foodborne illnesses, C. jejuni is also a primary etiological agent for ruminant abortion (14). Recently, we reported the emergence of an antibiotic-resistant and hypervirulent clone of C. jejuni in the U.S. (15). It is named clone SA (for sheep abortion) and is responsible for the majority (>90%) of ovine abortion cases in the U.S. (15). The clonality of SA strains was established by multiple molecular typing methods including pulsed field gel electrophoresis and multilocus sequence typing (ST-8) (15). Notably, C. jejuni clone SA is zoonotic and has been implicated in a number of outbreaks and sporadic cases of foodborne illnesses in humans (16). Clone SA infection is characterized by its extraordinary ability to translocate across the intestine, induce systemic infection, invade the uteroplacental unit with high titer, and cause abortion in pregnant animals as compared with other strains of C. jejuni (15, 17).

Epidemiological analysis of historical clone SA isolates suggested that its emergence in the U.S. is likely to be a recent event(15), but the genetic basis for its emergence and enhanced virulence is unknown. To facilitate understanding the pathogenesis, we sequenced a representative clone SA isolate (IA3902), which revealed that the genome of IA3902 is highly syntenic to that of C. jejuni strain NCTC11168 (18). Importantly, C. jejuni NCTC 11168 was shown to be non-abortifacient in pregnant animals(17), providing a closely related control strain for elucidating the hypervirulence of clone SA. Comparative genomics revealed numerous genetic differences between the genomes of IA3902 and NCTC 11168, including 57 unique genes and >8,000 single nucleotide polymorphisms (SNPs) as well as small (<10 bp) insertions and deletions (Indels). Transcriptomic and proteomic analyses identified multiple differences in the expression of genes involved in several classical virulence-related pathways: iron acquisition, capsule biosynthesis, energy metabolism, and motility (18). However, the causative genetic changes driving clone SA's success in causing disease remained undetermined.

In this study, we integrated genomics with experimental approaches to reach beyond correlative analyses to identify the exact genetic changes responsible for hypervirulence. We first sequenced a panel of C. jejuni strains, which established the emergence and rapid expansion of C. jejuni clone SA across the United States. Next we developed the “directed genome evolution” strategy, which takes advantage of transformation between two genetically similar but phenotypically different (abortifacient and nonabortifacient) strains, positive screening in an animal model of the hybrids that gain virulence, and whole-genome sequence analysis to pinpoint the locus and mutations responsible for the disease phenotype. This strategy effectively identified mutations in a single causative gene (porA; encoding the major outer membrane protein) driving the abortion phenotype of clone SA. Subsequent experiments verified that point mutations in a single, surface-exposed loop (loop 4) of the major outer membrane protein were both necessary and sufficient for causing abortion in the guinea pig model. Armed with this definitive genetic evidence, we evaluated several computational tests for adaptive evolution using the genome sequencing data, finding that Fu's Fs test singled out porA as the gene with the strongest genome-wide signal for adaptive evolution. This represents a rare example of arguing that genome sequence analysis can, in an unbiased fashion, produce clear hypotheses for causative genetic changes.

### Results and Discussion

### Comparative Genomics and Verification of Clone SA Virulence

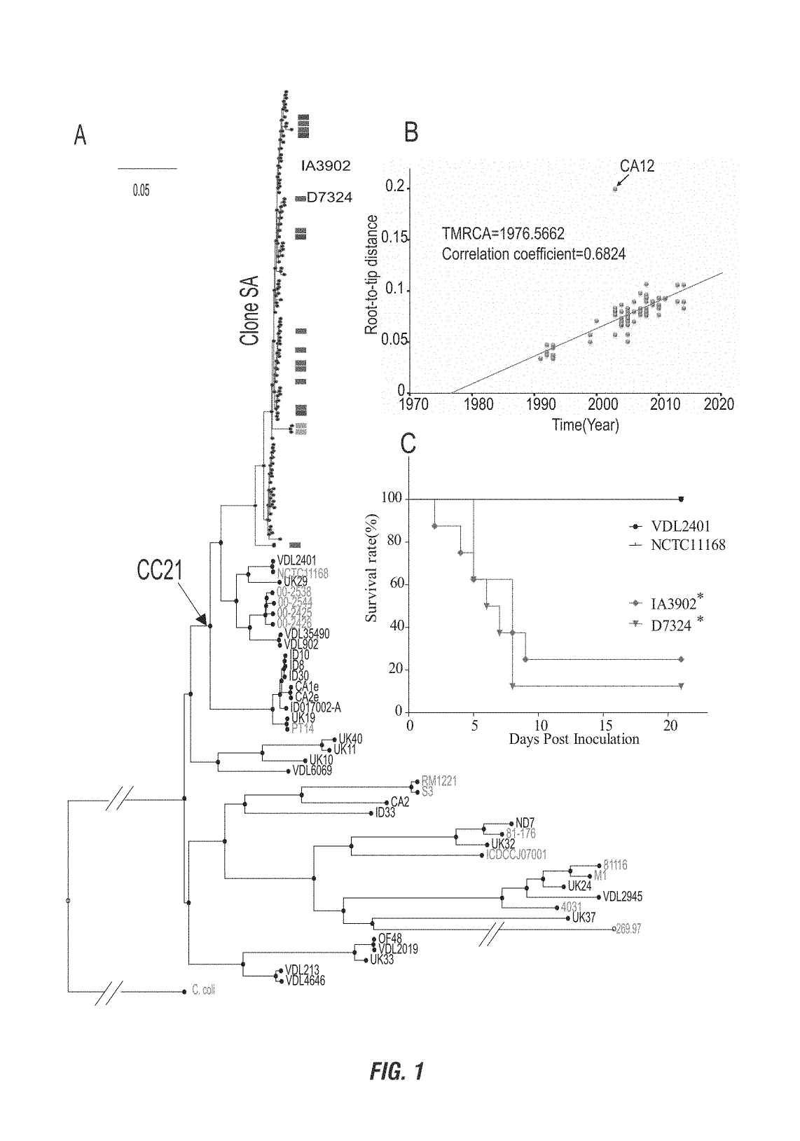

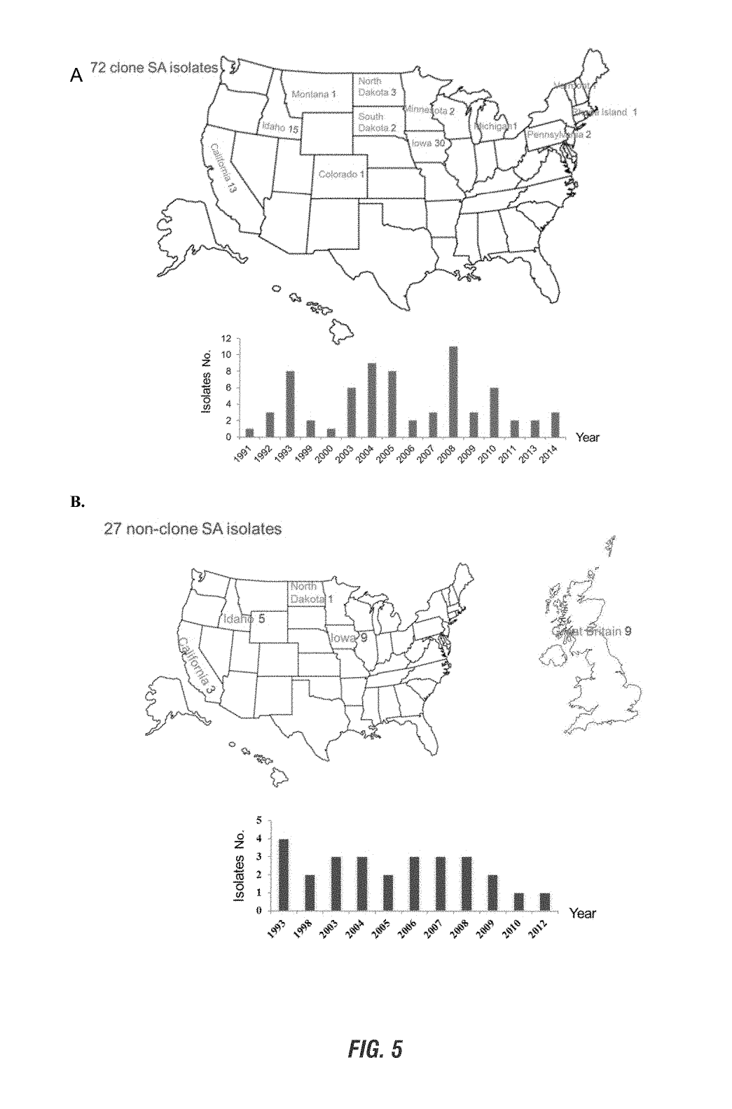

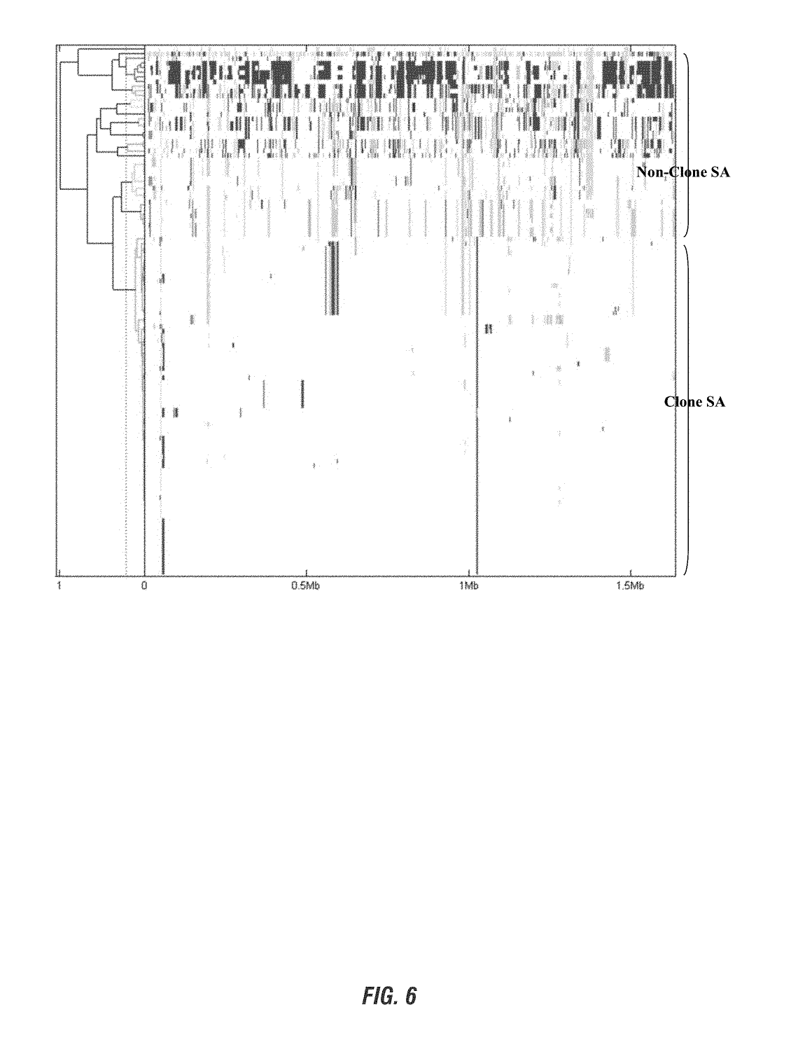

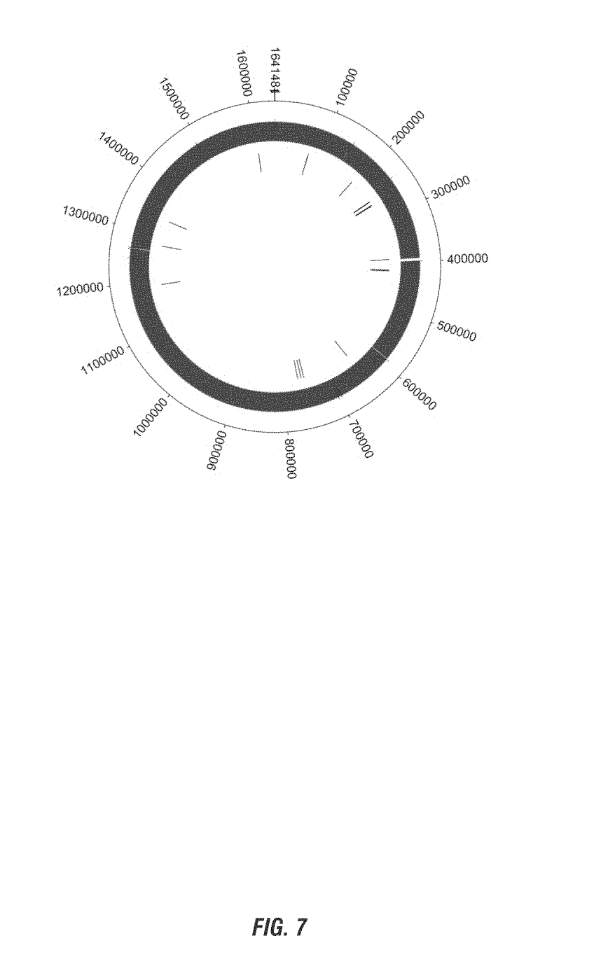

In total, ninety-nine C. jejuni isolates were sequenced, including 72 clone SA isolates (ST-8) and 27 non-clone SA isolates (non ST-8). They were selected from our collections over the last two decades to represent historical and contemporary isolates of clone SA as well as sporadic non-clone SA abortion isolates in the United States and Great Britain (Table A and FIG. 5). A whole-genome phylogeny definitively confirmed the previous MLST data (15, 16) that clone SA has undergone a recent, monophyletic clonal expansion into different hosts (cattle, goat, chicken, and human). This was supported by the tree topology (FIG. 1A), reduced recombination subsequent to emergence (FIG. 6), and a reduced haplotype diversity (median h=0 for clone SA versus 0.794 for non-clone SA isolates). In contrast, the non-clone SA abortion isolates are paraphyletic and genetically indistinguishable from non-abortion reference strains (FIG. 1A & FIG. 7). Molecular clock analysis led to an estimate of its emergence in mid 1970s (FIG. 1B). This finding is comparable with the time detected for the species shift from C. fetus to C. jejuni in the etiology of sheep abortion (15, 16), which could be due to the deployment of C. fetus vaccines. Additionally, we performed animal infection experiments using clone SA and non-clone SA isolates, which showed IA3902 and D7324 (clone SA isolates) were highly abortifacient, while NCTC11168 and VDL2401 (non-clone isolates) did not induce any abortion in the inoculated guinea pigs (FIG. 1C). The differences in abortion rates were statistically significant (p<0.05). These findings are consistent with the results from previous studies(17) and demonstrated the distinct ability of clone SA in abortion induction. The enhanced virulence of clone SA in inducing abortion has likely facilitated its transmission in sheep flocks as aborted materials (fetus, placenta, uterine discharge, etc) serve as an important source of infection for healthy ewes. Thus, the hypervirulence is not only a marker for clone SA, but may also be linked to its emergence as the predominant cause of sheep abortion in the U.S.

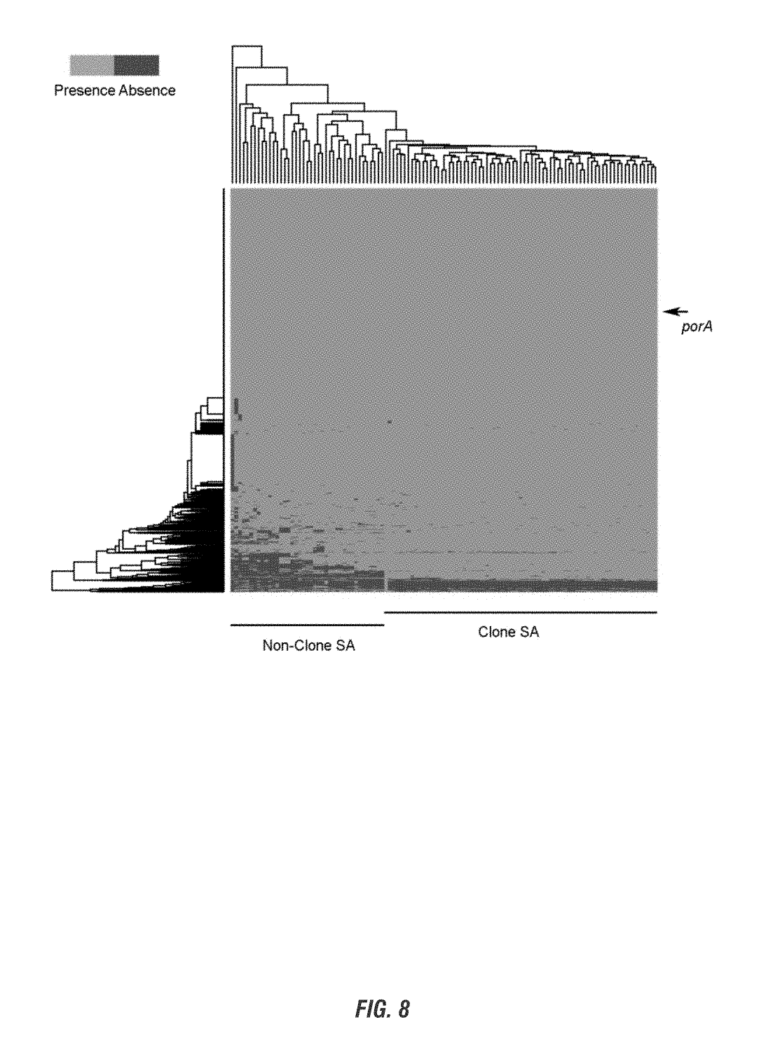

The monophyletic emergence of clone SA and its ability to induce abortion is thought to have been due to the ultimate acquisition of genetic changes enabling its virulence. Such changes could involve either small SNP/Indels or larger events such as gene gain/loss due to horizontal gene transfer or chromosomal rearrangements. However, our initial effort focusing on examining gene contents by comparing clone SA with non-clone SA isolates did not identify plausible candidate genes that were unique to clone SA and might explain the hypervirulence (FIG. 8). Thus, the causative loci responsible for the hypervirulence of clone SA remained unknown.

### Design of a Directed Genome Evolution Strategy for Identifying Causative Mutations

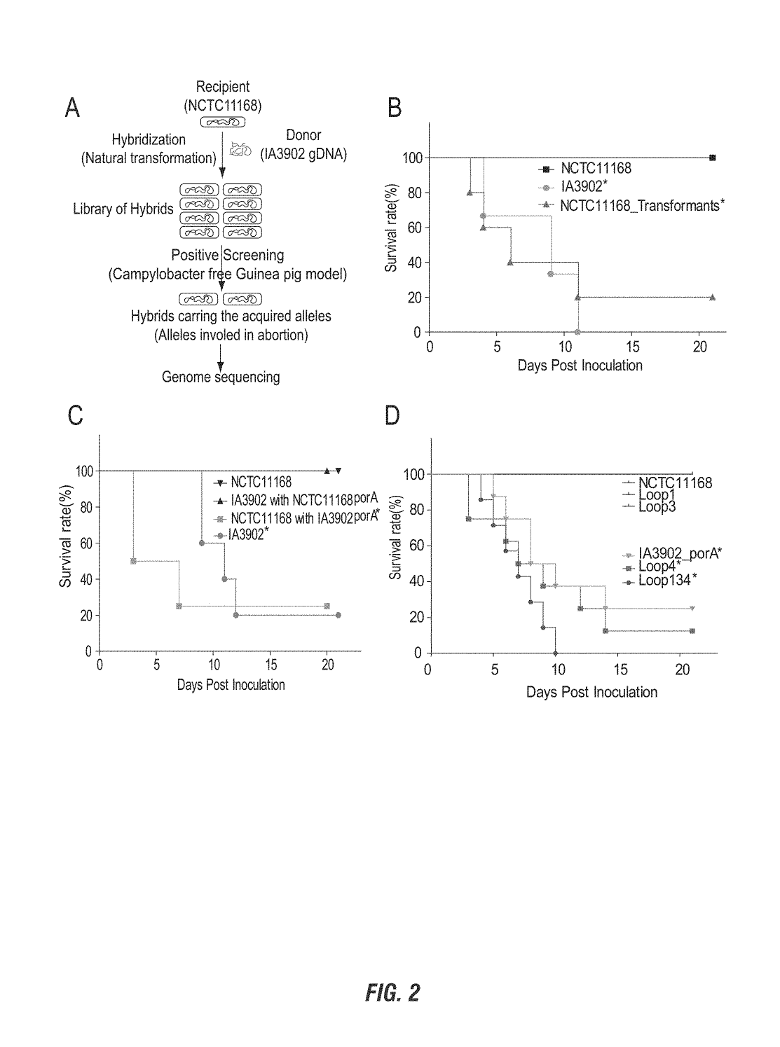

We therefore devised an experimental strategy to identify the genetic changes causing abortion. This strategy involved generation of hybrids by natural transformation between two genomically similar, but phenotypically different C. jejuni strains, positive screening in an animal model of the hybrids (transformants) that gained virulence, and whole genome sequence analysis of the hybrids from infected tissues (FIG. 2A). We termed this strategy “directed genome evolution” as it mimics genome-wide evolution guided by positive selection. The transformation was performed between C. jejuni NCTC11168 and a clone SA isolate IA3902, which were non-abortifacient and highly abortifacient, respectively, as demonstrated in a guinea pig model (FIG. 1C)(17). Purified genomic DNA of strain IA3902 was used as the donor DNA, while C. jejuni NCTC11168, which has a genome highly syntenic to that of IA3902(18), was used as the recipient strain. Complete genome sequences are available for both strains, but 25 chromosomal genes and an extra plasmid are found to be specific for IA3902; in addition, 8,696 SNPs and Indels were identified between these two strains (18, 19). Thus, individual analyses of the contribution of these differences to virulence would be time- and cost-prohibitive, which demanded for an effective and high throughput strategy for screening the candidate loci.

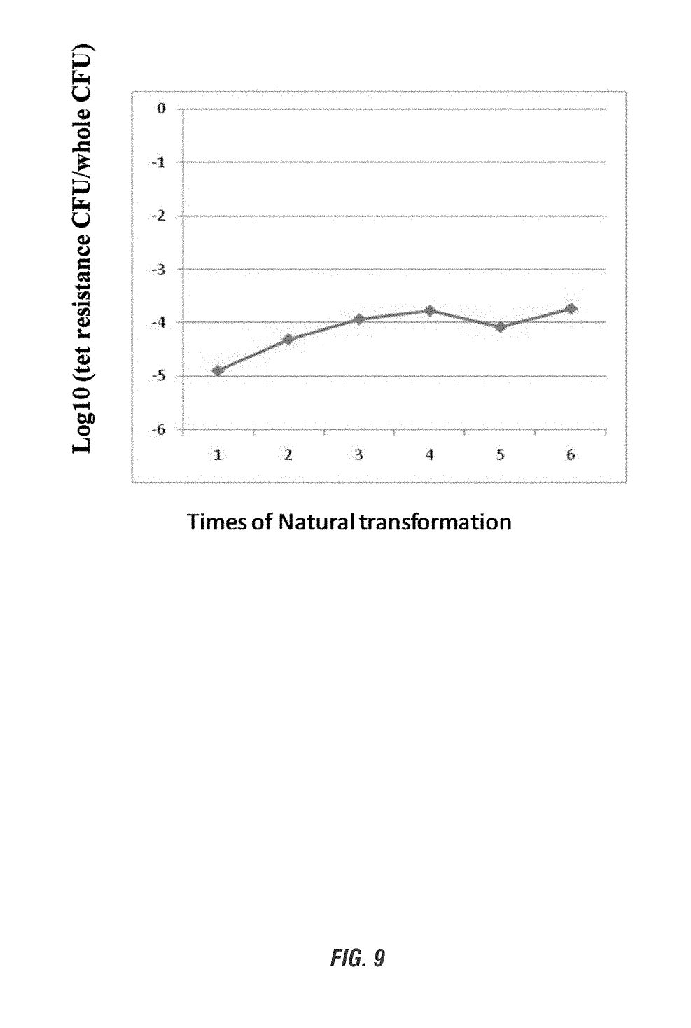

Non-abortifacient NCTC 11168 was grown through serial passages in culture media in the continuous presence of purified IA3902 (abortifacient) genomic DNA. Transformation efficiency was monitored by measuring the frequency of tetracycline resistant colonies. The recipient NCTC11168 is tetracycline susceptible, while IA3902 is resistant due to the possession of a chromosomal tet(O) gene (18). The frequency of tetracycline resistant transformants plateaued after 4 passages (FIG. 9), and this library of transformants was used for inoculation of guinea pigs.

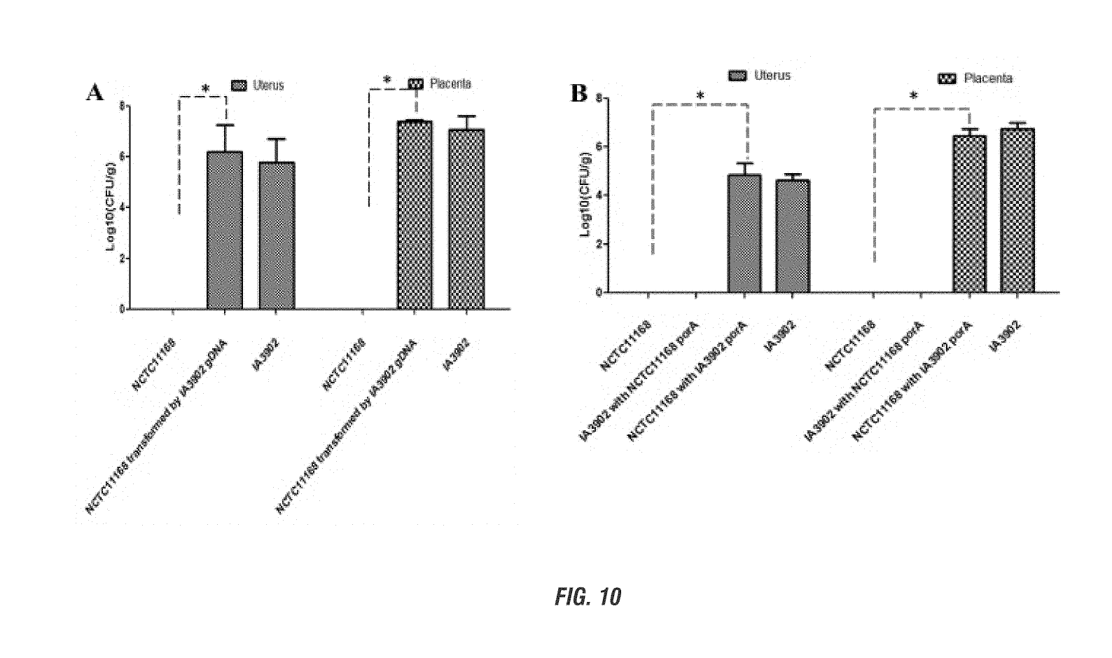

Via oral gavage, three groups (n=8 per group) of guinea pigs were inoculated with NCTC11168, IA3902, and the library of NCTC11168 transformants (hereafter NCTC11168-tr), respectively. Abortion in this model is associated with the ability to translocate across the intestinal barrier and induce infection of the uterus and placenta(17). The non-abortifacient NCTC11168 colonized the intestinal tract, but did not induce abortion, histological signs of inflammation in uterus and placenta, or infection of the uteroplacental unit as confirmed by bacterial culture. In contrast, animals infected with IA3902 or NCTC11168-tr yielded high and comparable (p>0.05) abortion rates (FIG. 2B), with high bacterial loads (FIG. 10A) and inflammation in uteroplacental units. This result clearly indicated that NCTC11168-tr gained the ability to induce abortion in pregnant guinea pigs. This finding was significant as it suggested that the virulence-contributing genetic factor was successfully transferred from IA3902 to NCTC 11168 via natural transformation.

Bacteria were isolated from the uterus and placenta of NCTC11168-tr-infected animals that had undergone abortion. We selected 12 individual isolates randomly picked from 3 aborted animals (4 per animal) as well as a pooled sample consisting of all remaining colonies from the uterus and placenta of all three animals. These isolates were from infected uterus and placenta and should have gained the virulence for abortion induction. This positive screening step in the guinea pig model ensured that those transformants that did not gain virulence were blocked entering the uteroplacental unit. The positive selection was amazingly effective as plating with Tet-containing media demonstrated that the input transformant library (˜109 CFU) contained a number of Tet-resistant colonies (˜105 CFU), while the pooled isolates from the infected uterus and placenta did not contain any Tet-resistant colonies. This indicates that the transformants which acquired tet(O) did not reach to uterus and placenta, consistent with the fact that tet(O) doesn't contribute to abortion induction, and also suggesting that tet(O) is not linked with a critical virulence factor required for abortion induction.

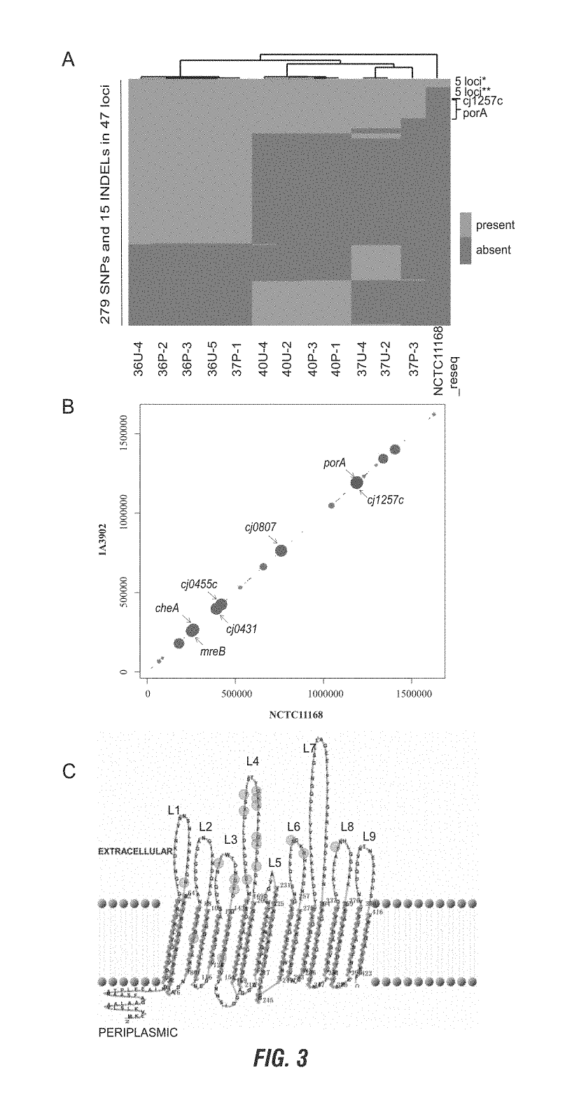

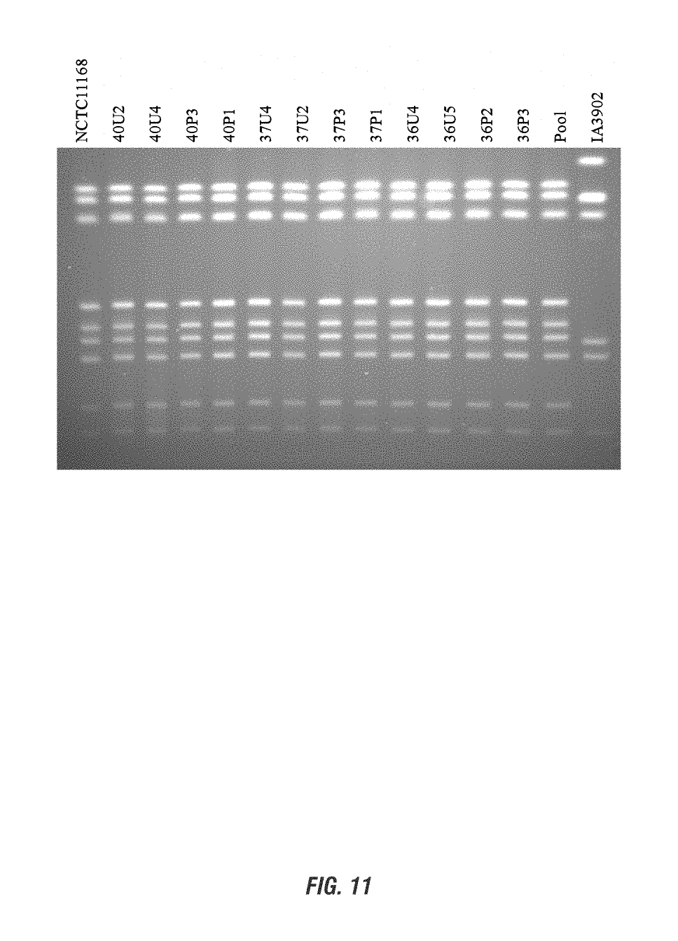

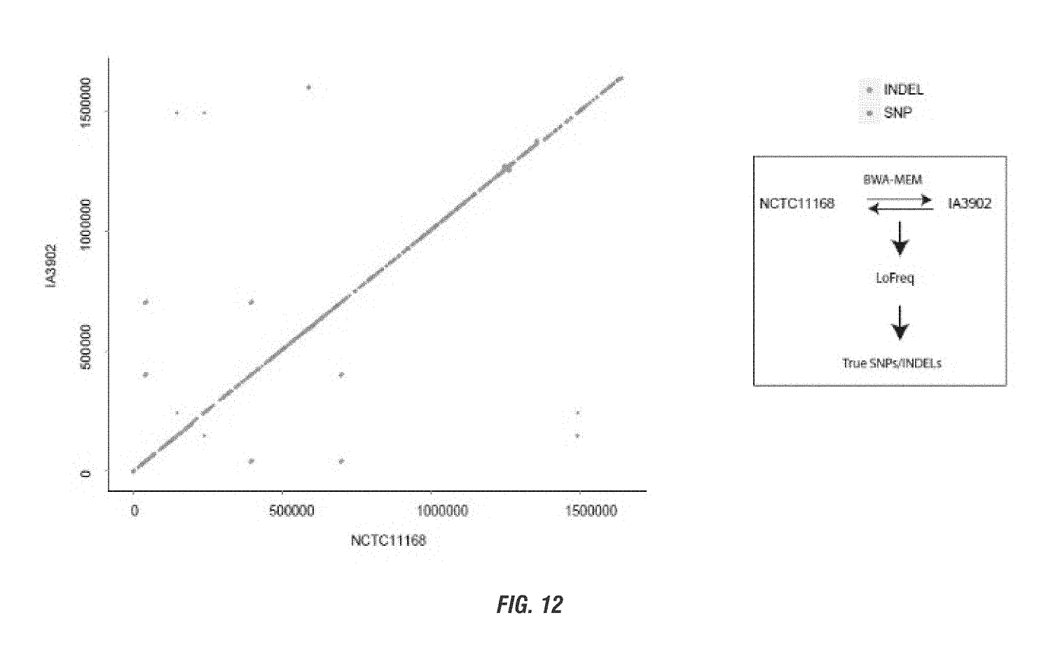

Pulsed field gel electrophoresis (PFGE) analysis demonstrated that the pool and all 12 individual isolates from the infected uterus and placenta were indeed NCTC11168-derived, as their PFGE patterns were identical to NCTC11168 (FIG. 11). This PFGE result also suggested that no large chromosomal rearrangements occurred in the transformants. The individual isolates and the pool were subject to whole genome sequence analysis, which did not reveal transfer of IA3902-specific genes to the selected isolates. Then we focused on analysis of the transferred small mutations (SNPs and Indels) using a conservative strategy (FIG. 12), in which only reads supporting differences between the two genomes that could be unambiguously identified were used. In all samples, as expected, most of the positions carried the original NCTC11168 (non-abortion) allele. Among the 12 individual isolates, a total of 47 loci representing 279 SNPs and 15 Indels (SNP/Indels from either genic/intergenic regions) were transferred in at least one isolate. In particular, 12 loci were unanimously present in all 12 isolates (FIG. 3A). However, the mutations in 5/12 loci (mreB, cheA, cj0431, cj0455c, cj0807) were also present in the parental NCTC11168 as re-sequenced in this study (FIG. 3A). Interestingly, in the pooled sample, only 7 out of these 12 loci had high allele frequencies (>99%) for the IA3902 (abortion) allele, including porA, cj1257c, and the 5 loci described above (FIG. 3B). The mutations in the remaining 5 loci did not have high frequencies in the pooled sample, though they were present in all 12 individual isolates (FIG. 3A). Therefore, only 2 loci, which contained two genes: cj1257c and porA, had high allele frequencies in both the individual colonies and the pooled sample, and did not have the corresponding mutations in the parental NCTC11168. cj1257c differs between IA3902 and NCTC 11168 by a synonymous SNP, while porA carries 16 synonymous and 25 nonsynonymous SNPs that lead to 18 amino acids substitutions (Table B). Therefore, our linkage analysis produced porA as the single most likely candidate gene for abortion. porA (cjsa_1198) and tet (O) (cjsa_0193) are distantly separated (by ˜990 Kb) in the chromosome of IA3902 and are unlikely co-integrated into the recipient during transformation, which further explains why tet(O) was absent in the transformants isolated from the uteroplacental units of aborted animals.

Specific Mutations in porA are Required for C. jejuni Systemic Infection and Abortion

We further validated the findings from the directed genome evolution experiment by using defined mutagenesis and animal studies. First, directed allelic exchange was used to swap the chromosomal allele of porA in IA3902 and NCTC 11168. IA3902 with the porA from NCTC1168 completely lost the ability to induce abortion, while NCTC11168 with the porA of IA3902 fully gained virulence in pregnant guinea pigs (FIG. 2C). In fact, the abortion rates between IA3902 and NCTC11168 with the porA of IA3902 were comparable and were not significantly different (p>0.05). This result showed that the IA3902 porA allele was both necessary and sufficient for causing abortion in the context of existing genes of NCTC 11168 (FIG. 2C).

porA encodes the major outer membrane protein (MOMP) in C. jejuni and MOMP has been shown to function as a porin and adhesion (20-22). MOMP is the most abundant membrane protein in C. jejuni with 18 β-strands and 9 external loops (23). The 18 amino acid differences between the porA alleles of NCTC11168 and IA3902 clustered preferentially on several of the 9 surface-exposed loops (FIG. 3C). We hypothesized that some of these mutations were required for abortion induction. To test the hypothesis, site-specific mutagenesis was used to introduce specific mutations into the loops of porA in NCTC11168. These mutant constructs were evaluated in the guinea pig model, which demonstrated that among the sets of mutations in loops 1, 3, and 4, only loop 4 mutations (containing 9 amino acid substitutions) were required and sufficient to confer an abortion phenotype when introduced into NCTC11168 (FIG. 2D). Again, in these experiments abortion was strictly correlated with C. jejuni infection in the uterus and placenta (FIG. 10b) and histopathological signs of inflammation. The mutations in loop 6 and loop 8 were not tested because they have low frequencies among either the transformants or the field clone SA isolates. These findings indicate that allelic variations in loop 4 are critical for the hypervirulence of C. jejuni clone SA.

porA Drives the Recent Expansion of Clone SA

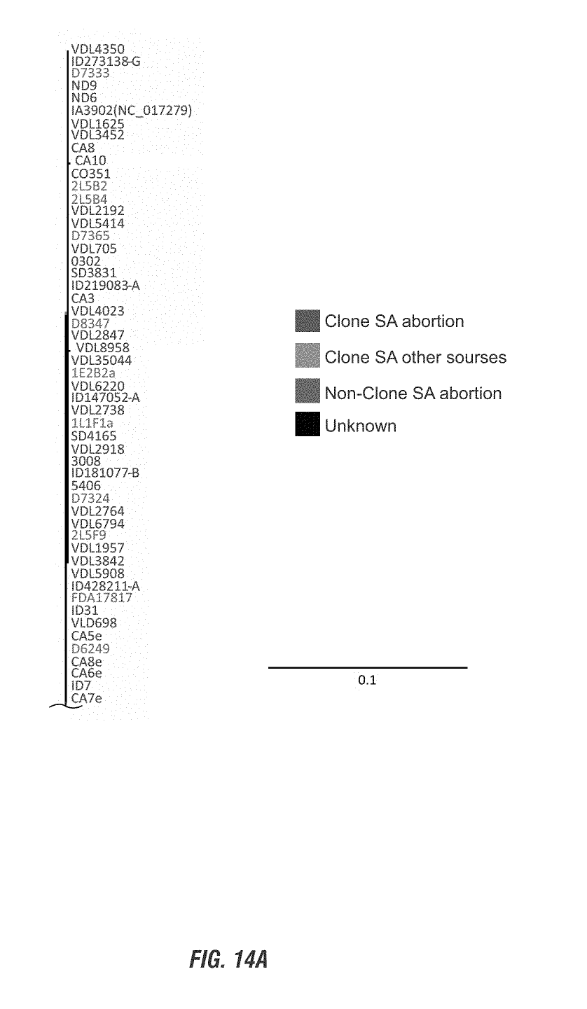

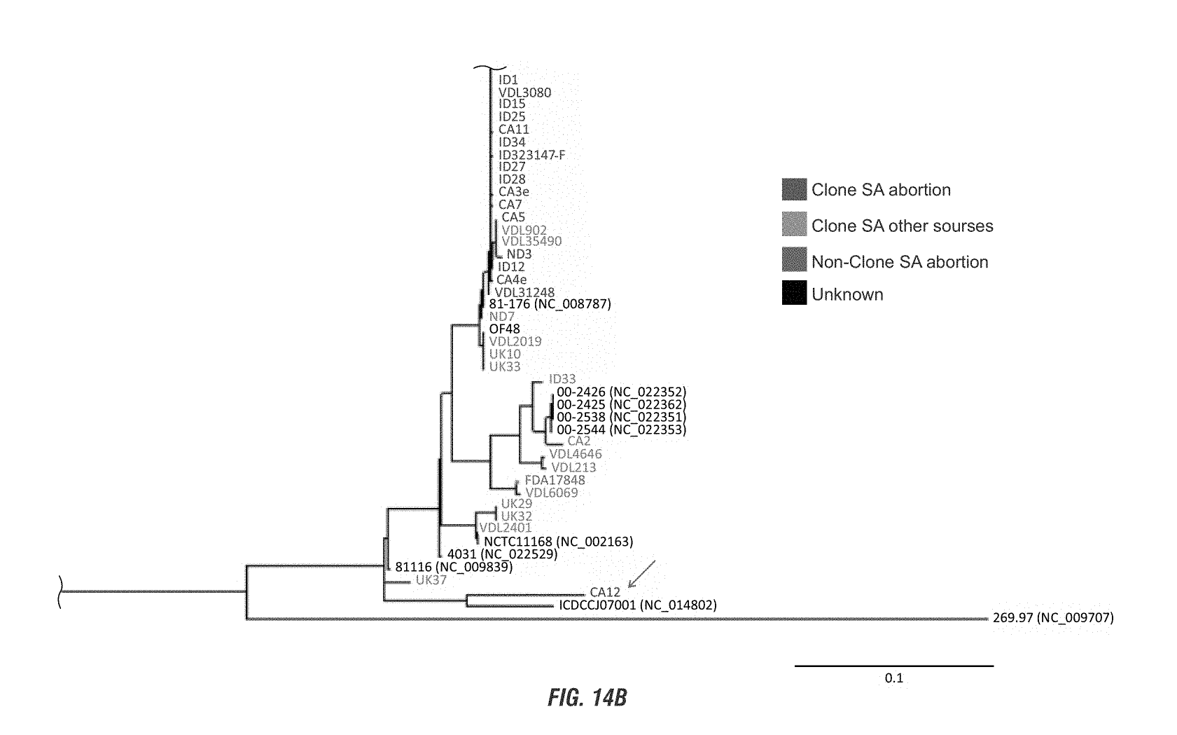

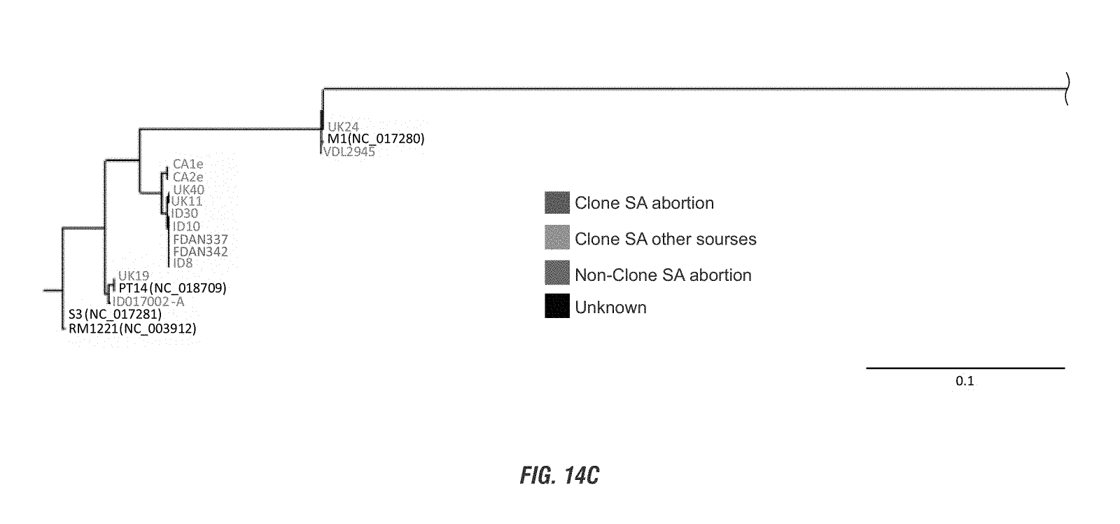

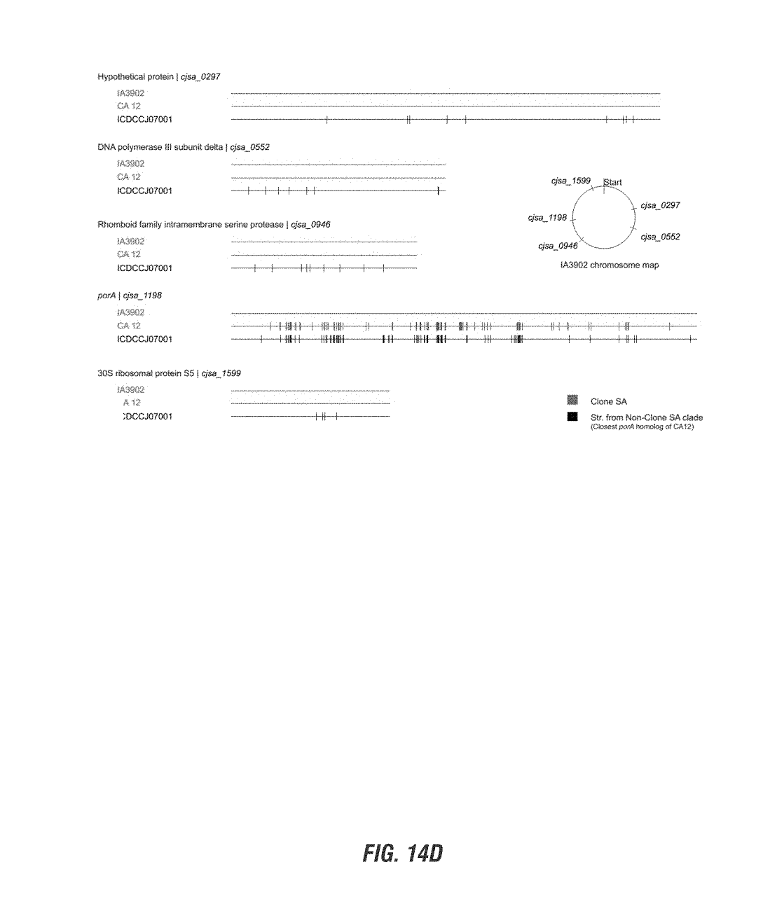

The porA alleles among clone SA isolates were closely related to the IA3902 allele (FIG. 14A-C). Strikingly, two non-clone SA strains (VDL902 and VDL35490), which were isolated from abortion cases, carried porA alleles very similar to clone SA strains. In addition, 4 clone SA isolates carried porA alleles hypervirulent from majority of the clone SA strains. 3 of them (FDA17848, FDAN337, and FDAN342) were isolated from non-abortion cases; but the other one CA12 was an exception, which was from a bovine abortion case and have two ˜37 Kb phage inserts in the genome compared to IA3902 and other clone SA strains. Although CA12 is categorized as a clone SA isolate based on its core genome, it is quite hypervirulent from other clone SA isolates (FIG. 1B). Additionally, detailed analysis of the porA allele indicated porA of CA12 was quite different from that of clone SA, but was closely related to that of ICDCCJ07001 (a non-clone SA strain) (FIG. 14), suggesting that recombination might have changed the porA allelic profile of CA12. These differences in the porA allele and whole genome phylogeny are likely due to recombination, which occurs commonly in Campylobacter (24, 25), and suggest that CA12 is an outlier of the clone SA clade.

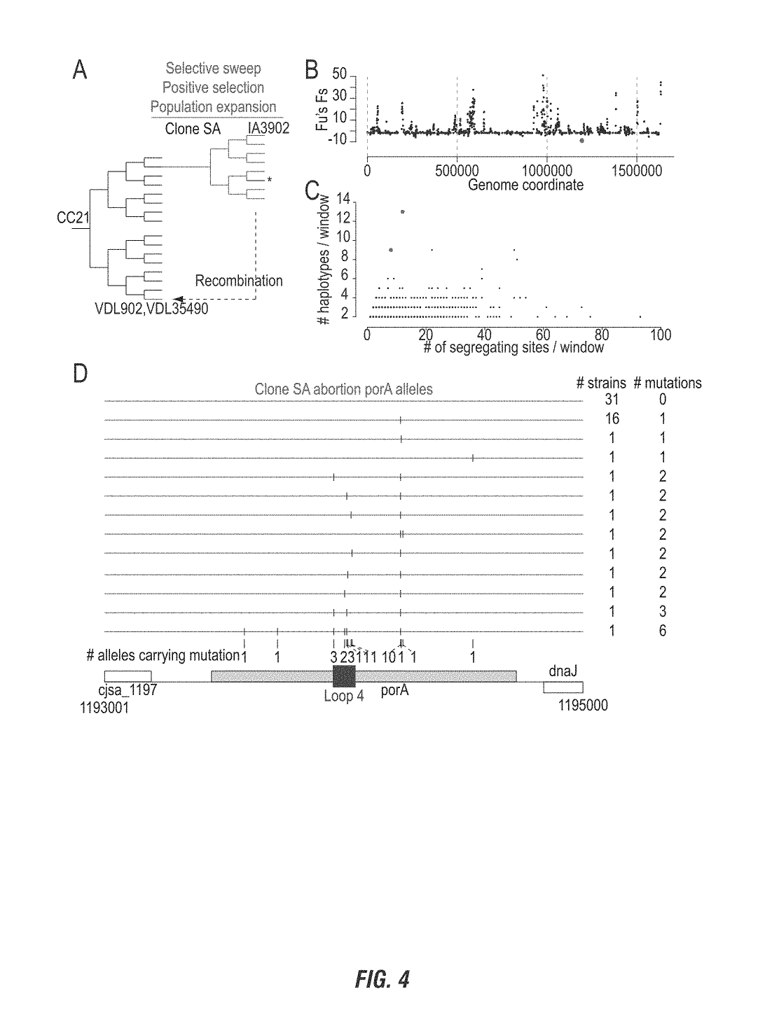

Genome sequencing is widely used for studying evolution of bacterial pathogens(8, 26). The finding that allelic variation in loop 4 of porA is necessary and sufficient for conferring abortion to NCTC11168 presents a unique data set in the sense of a positive control for evaluating sequence analysis methods that can directly provide mechanistic insights into disease. The epidemiology of clone SA suggests a recent selective sweep (driven by positive selection) acting on the porA gene (FIG. 4A). Several tests are designed to detect signals of recent selective sweeps or positive selection from sequence data (27-30). However, neither selective sweep nor codon-based positive selection tests found a signal at the porA locus that would stand out from all other genes in the genome. Thus these methods were unable to find a signal associated with abortion for porA (see Supplementary Results).

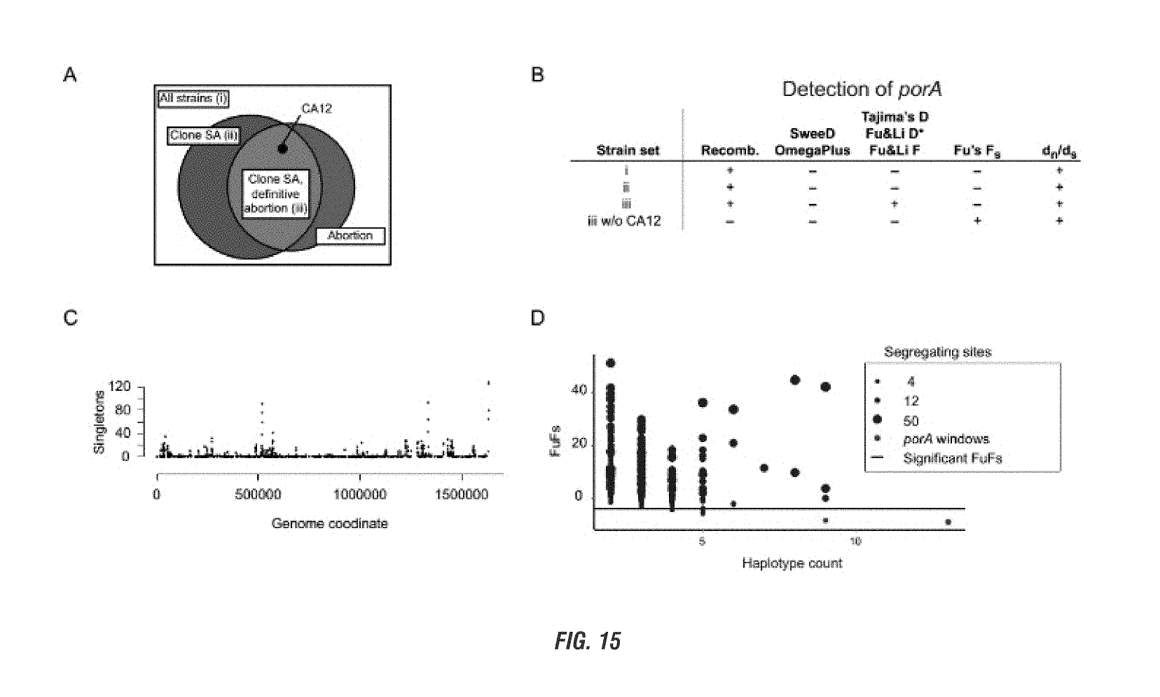

Because we sampled strains from different time periods and geographic locations, which might violate demographic assumptions of some population genetics analyses(31), we focused our analysis solely on clone SA isolates from known abortion cases. We reasoned that this would enable analysis on the relevant population after the selective sweep and population expansion and also remove the confounding effect of recombination at the porA locus. As noted above, among the 59 clone SA isolates known from abortion cases, one strain, CA12, had a hypervirulent porA, likely due to recombination (FIG. 14). Therefore, we performed analysis on data sets including and excluding CA12. Strikingly, in the non-recombined data set (excluding CA12), Fu's Fs test predicted porA as the top locus driving selection/population expansion (FIG. 15B; see Supplementary Results for full details of analysis of all data sets). A typical signature of a recent selective sweep is an enrichment of recent, rare mutations that result in an excess of unique haplotypes at the selected locus (30, 32, 33). Indeed, we saw precisely this theoretical pattern at the porA locus of clone SA, centered on loop 4, explaining its outstanding signal using Fu's Fs test (FIG. 4B-D). Inclusion of CA12 in the analysis would falsely increase the overall diversity of a clonally expanding population without an apparent increase in haplotype frequency. As Fu's Fs test relies on haplotype frequency given the diversity of the population (30), this would result in a false negative signal for porA, justifying the exclusion of CA12 from the analysis.

Our finding of porA as the top hit using Fu's Fs provides a rare example suggesting that genomic analysis may be capable of directly identifying disease-causing loci in silico. This relied on outstanding metadata regarding abortion; clear epidemiology indicating population expansion; and an experimentally verified data set on porA provided by the directed genome evolution strategy. We were able to manually examine our data set for recombination due to the knowledge that mutations in porA were responsible for abortion, underlying the need for better methods to automatically remove such confounding factors in the prediction of unknown loci. The ability of Fu's Fs for identifying the genetic basis for clone SA's success matches expectations gained from theory and simulation(30, 34, 35), indicating that further developments in characterizing recombination, population growth models, and sampling strategies could have broader impact on the value of other pathogen sequencing projects.

### Concluding Remarks

Emergence of pathogenic variants is often driven by their increased virulence or fitness, possibly associated with the gain of novel genes or mutations(1). Identification of the genetic basis for altered virulence phenotypes is essential for developing better strategies to recognize, predict, and control disease emergence and epidemics(36). In recent years, development of next-generation sequencing technologies has greatly facilitated our understanding of pathogen evolution as well as genotype-phenotype relationships. In many cases, investigation focused on large regions of genetic variation, such as pathogenicity islands, recombination events, and mobile genetic elements that introduce novel genes to emergent and epidemic strains(1). Recent examples include acquisition of superantigens SSA and transposable elements encoding multidrug resistance genes driving the expansion of scarlet fever-associated Streptococcus pyogenes emm12 lineages in Hong Kong (5) and horizontal transfer of a mobile genetic element-encoded gene sasX driving epidemic waves of methicillin-resistant Staphylococcus aureus (MRSA) infection(4). While SNPs are the most common sources of genomic variations within bacteria and also involved in the change of pathogen's virulence and host tropism(37-40), such mutations are rarely reported to drive clonal expansion of bacterial pathogens. It is likely that the sheer number of SNPs existing among strains of the same bacterial species, coupled with the paucity of methods for effective screening of causative mutations, has led to this bias towards large gene and mobile element differences in existing genomics studies.

In this study, we have discovered that specific amino acid substitutions in loop 4 of MOMP encode by porA are responsible for the hypervirulence of C. jejuni clone SA. How these minor changes lead to a major change in virulence is intriguing, but it is probably due to alteration of a key function of MOMP, which has been identified as a porin and adhesion(20-22). Additionally, glycan modification of MOMP was previously shown to be involved in intestinal colonization (20). However, these previously known functions of MOMP may not fully explain its key role in causing abortion and it is highly possible that MOMP has a yet-to-be-discovered role in mediating pathogen-host interaction and enabling systemic infection. This possibility remains to be determined in future studies

Work presented in this study represents the first report documenting the key role of MOMP in systemic infection and abortion induction by C. jejuni. This finding is significant as it provides a potential target for controlling the spread of C. jejuni clone SA, which is a zoonotic pathogen causing abortion in ruminants and foodborne illnesses in humans(15, 16). Additionally, we developed a “directed genome evolution” strategy for effective screening of causative mutations, which may be applied to understand the virulence mechanisms of other pathogenic organisms that are amenable for the same strategy. Compared to traditional loss-of-function, gain-of-function, or random mutagenesis screens, this strategy has the primary advantage of being efficient and adaptable. By starting with phenotypically different strains, the causative genetic changes responsible for a phenotype can be readily identified using the strategy. Furthermore, our data present a unique case study for evaluating and refining methods used for genomic analyses. Finally, findings from this study provide a new direction for investigating pathogen-host interactions during infection by C. jejuni.

### Materials and Methods

Bacterial Isolates

Ninety-nine C. jejuni isolates, including their time and location of isolation, are listed in Table A and FIG. 5. Clone SA strains (n=72) were initially classified by PFGE and MLST analyses (ST8)(1, 2). They were obtained from aborted fetoplacental tissue of sheep (n=49), goats (n=4), and cattle (n=5) as well as from non-abortion associated sheep feces and bile (n=5). In addition, several clone SA isolates (n=9) provided by the US FDA and CDC were also included in this study(2). Non-clone SA isolates (non-STB, n=27) are from sporadic sheep abortion cases in the United States and Great Britain.

C. jejuni IA3902 (a prototypical isolate of clone SA) is a clinical isolate obtained in 2006 from an aborted sheep placenta and was shown to be highly virulent in inducing abortion in a pregnant guinea pig model (1, 3). C. jejuni NCTC11168 was originally isolated from a diarrheic patient and was previously demonstrated to be non-abortifacient in pregnant animals(3). Both IA3902 and NCTC11168 have been sequenced and were used in this study as references(4, 5).

Whole-Genome Sequencing

Genomic DNA was prepared using the Wizard Genomic DNA kit (Promega) according to the manufacturer's instructions. Indexed Illumina sequencing libraries were prepared using TruSeq DNA PCR-Free Sample Preparation Kit (Illumina Inc., San Diego, Calif.) following the standard protocol and sequenced as single-end or paired-end runs on the Illumina HiS eq 2000 or Illumina MiS eq platform according to the manufacturer's protocols. At least 100× coverage of raw reads were yielded for each isolates. Reads data were submitted to the Sequence Read Archive (SRA) under accession SRP067965 (the full list of accession codes is given in Table A)

Phylogenetic and Temporal Analyses

A whole genome SNP phylogeny was constructed by direct mapping of reads to the reference sheep abortion strain IA3902 (RefSeq Accession No. NC_017279). The dataset included 99 clinical isolates (72 Clone SA and 27 Non-Clone SA) and 15 publicly available curated C. jejuni genomes (including the above reference sheep abortion strain IA3902, and 14 other Non-Clone SA). The tree was rooted to a C. coli CVM N29710. Altogether 115 genomes were used in this analysis (Table A). Simulated reads for the genomes from NCBI database were created using wgsim (distributed with SAMtools)(6). All the read sets were aligned with the Burrows-Wheeler Aligner (BWA)(7) against the genome sequence of strain IA3902. SNPs were identified with SAMtools. Repetitive regions in the reference genome sequence were characterized using the repeat finder functions in the MUMmer package(8). SNPs falling within these repetitive regions were excluded from analysis. A maximum-likelihood phylogenetic tree was constructed from SNP alignment with FastTree (v2.1.8)(9) using a general time-reversible model. The phylogenetic tree was visualized with FigTree (http://tree.bio.ed.ac.uk/software/figtree). The BratNextGen method (10) was used to detect recombination events using the sequence alignment obtained above. After the removal of recombinant segments, mobile elements and repetitive sequences, the temporal signals in the sequence data of clone SA were analyzed using Path-O-Gen (http://tree.bio.ed.ac.uk/software/pathogen/).

Gene Content Analysis.

Gene content profiling included 99 clinical isolates and 15 reference C. jejuni genomes as mentioned above for phylogenetic analyses. Reads were assembled using the de novo short-read assembler Velvet(11) and Velvet Optimiser (http://bioinformatics.net.au/software.velvetoptimiser.shtml). CDSs were predicted from each draft genome sequence using default parameters in Prodigal(12). Orthologous groups were computed using GET_HOMOLOGUES(13) with 90% coverage and 70% sequence identity as cut off. A binary matrix of gene presence and absence was computed using the bidirectional best-hit algorithm relative to IA3902 protein coding genes as the reference. The resultant matrix was then used to plot a heat map using heatmap.2 function in R package, gplots (http://CRAN.R-project.org/package=gplots). Overall, Clone SA and non-clone SA formed 2 distinct clades. Genes which were previously known to be unique to representative (NCTC11168) and abortion (IA3902) strains were re-capitulated(1). However, no unique signatures to either of the groups represented by the above representative strains were identified.

Construction of NCTC11168 Transformants

Genomic DNA was extracted from IA3902 of C. jejuni clone SA using the Promega genomic DNA purification kit (Madison, Wis.). The transformants were made by natural transformation of IA3902 gDNA into NCTC11168 (please note that wild type NCTC11168 is non-abortifacient clinically and in animal model) on Mueller-Hinton (MH) agar as described previously(14). Of note, IA3902 is tetracycline resistant conferred by chromosomally-encoded tetO gene whereas NCTC11168 is tetracycline susceptible. Tetracycline resistance of transformants was tested on MH agar with 20 μg/ml tetracycline hydrochloride (Sigma-Aldrich Co. LLC.).

Guinea Pig Abortion Model

All animal procedures were conducted in accordance with NIH guidelines, the Animal Welfare Act and the U.S. federal law. All animal experiments were performed with protocols approved by the Institutional Animal Care and Use Committee at Iowa State University (IACUC).

Palpably-pregnant (c.a. at 35 days of gestation) Hartley guinea pigs were obtained from a commercial source (Elm Hill Labs, Chelmsford, Mass.) for use in the study. At arrival, a rectal swab specimen was obtained from each guinea pig, and plated onto MH agar with Campylobacter-selective supplements (SR0232E and SR0117E; Oxoid) for Campylobacter-free confirmation. Animals were housed individually in cages with wood chip bedding and fed with commercial food pellets formulated for guinea pigs. Within 48-h of housing, the animals were subjected to ultrasound scan to re-confirm pregnancy and to prevent a potential disproportionate distribution of non-pregnant animals among groups as the commercial source typically provides only about 85% of the guinea pigs being pregnant at this stage of gestation. For the oral challenge exposure, each group included eight animals and inoculated with a different C. jejuni strain. Strict hygienic procedures were employed during the entire experiment to prevent cross-contamination between different groups. Animals were sacrificed if there were any signs of abortion (i.e., vaginal bleeding and/or expelled fetuses) and uterus as well as placental tissues were homogenized and plated onto Campylobacter-selective MH agar for culture. In addition, maternal bile, blood, intestinal content as well as fetal tissues were cultured for Campylobacter. At 20-21 days post of inoculation (dpi), all non-aborted animals were euthanized and examined for pregnancy status as well as culture as described above. In addition to culture, histopathology was performed on all of the uterus and placenta collected at necropsy. As described previously(3), no signs of infection were evident by either culture or histopathology in the uterus of non-pregnant guinea pigs. Thus, non-pregnant animals were excluded from statistical analyses of data. Statistical analyses were done using the GraphPad Software package (Graph-Pad Software, LaJolla, Calif.). Survival curves were compared by the log-rank (Mantel-Cox) test and Mann-Whitney U test was performed to compare the CFUs recovered from the tested samples between groups for statistical significance at p<0.05.

Genome sequencing and comparative genomics of abortifacient NCTC11168 transformants.

NCTC11168 transformants (NCTC11168-tr) with an apparent abortion were recovered from the uterus and placenta of aborted animals via culture on MH agar with Campylobacter-selective supplements. 12 individual transformants and the pool of all the remaining transformants from 3 aborted guinea pigs were sequenced as described above, and wild-type NCTC11168 was re-sequenced as control (NCTC11168-reseq).

Individual NCTC11168 tranformants were assembled de-novo using Velvet(11). Contigs were aligned to wild-type NCTC11168 genome (Genbank Accession No. NC_002163) using Mauve(15) to examine the gained genes from IA3902. Then reads of the transformants were mapped using BWA-MEM(7) to both of the NCTC11168 and IA3902 genome and duplicate marked using Picard 1.74 (https://sourceforge.net/projects/picard/files/picard-tools/1.74/). SNPs/Indels were called using LoFreq 2.1(16). To correct for potential mapping biases and repeated sequences, only variants that could be definitively called by short read sequencing were used in subsequent analyses. These definitive variants were determined by generating all 250 bp sub-sequences (by a sliding window of 1 bp) of NCTC11168 and IA3902, then passing these through the same BWA-MEM mapper against the other genome. Only those variants which were present at the same position in both comparisons were used (FIG. 12). Furthermore, some of the Indels that spuriously arose in coding sequences because of read alignment by BWA-MEM at low complexity regions were corrected using reverse translated-independent protein alignments of the corresponding coding sequences of the representative commensal and the abortion strains.

porA Allelic Exchange in IA3902 and NCTC1168

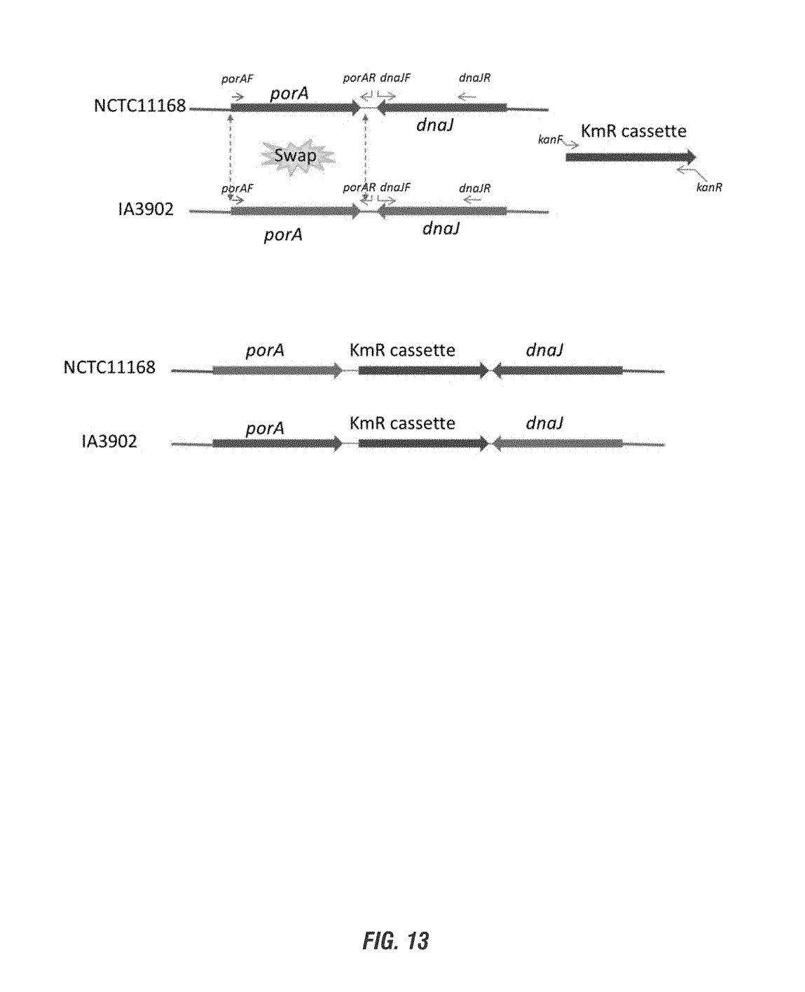

The entire porA gene of both IA3902 (cjsa_1198) and NCTC11168 (cj1259) was amplified by primers porAF and porAR; a part of the downstream gene dnaJ was also amplified by primers dnaJF and dnaJR from both strains. Kanamycin-resistance cassette (kan) was amplified by primers kanF and kanR from plasmid pMW10 (17). To adjoin porA, kan, and dnaJ, restriction site for BamHI was added to the primers porAR and kanF, and restriction site for PstI was added to the primers dnaJF and kanR. All three PCR products were digested separately by the corresponding restriction endonuclease(s) and ligated in a single reaction by T4 DNA ligase in such a way to generate either IA3902porA-kan-NCTC11168dnaJ or NCTC11168porA-kan-IA3902dnaJ. These three-fragment-ligated products were directly used as templates for PCR amplification by primers porAF and dnaJR. Each of the amplified products was then naturally transformed into NCTC11168 or IA3902 to facilitate the exchange of porA alleles between the wild type strains, generating either NCTC11168 carrying porA of IA3902 or IA3902 harboring porA of NCTC11168 (FIG. 13). The exchange of the porA allele in the constructs was confirmed by PCR and DNA sequencing. To define the virulence of mutations on external loops of IA3902 porA, entire porA gene variants of NCTC11168 with IA3902 Loop1, Loop3, Loop4, or Loops134 were synthesized in GenScript (Piscataway, N.J.), then ligated with kan-NCTC11168dnaJ PCR product, and introduced into NCTC11168 using the same strategy. All transformants were confirmed by PCR and Sanger sequencing of the porA gene to ensure the desired mutations were introduced into NCTC 11168. All the restriction endonuclease, T4 DNA ligase, high fidelity PCR enzyme Phusion were obtained from New England Biolabs (Ipswich, Mass.). Primer sequences and PCR conditions are listed in Table B.

Whole Genome Positive Selection Analysis

Protein sequences of each orthologous group constructed in gene content analysis were aligned using ClustalO(18). Protein sequences were then reverse translated guided by the corresponding nucleotide sequences. Haplotype diversity of clone SA isolates and non-clone SA isolates was compared by DNASP 5.10.1(19). These DNA alignments were then scanned for mutational changes that confer an adaptive advantage, using both molecular evolution and population genetic methods. To detect molecular evolution at codon level resolution, CDS alignments were initially screened for presence of recombination break points using a genetic algorithm for recombination detection (GARD(20) which was available as a part of HyPhy 2.2.4(21)). Potential break points predicted by GARD and the resultant tree topologies (e.g., before and after the break point) were statistically tested for topological incongruence using the HyPhy module GARDProcessor. CDS alignments and their corresponding phylogenetic trees were then used for detecting positive selection using maximum likelihood based, codem1 nested models, m8/m7 and m2a/m1a from PAML 4.5(22). For every CDS alignment, best likelihood for positive selection models m8 and m2a were calculated from running codem1 with 3 starting omega values (0.2, 1, 5) and their likelihood differences against nearly neutral models m7 and m1a were tested for statistical significance under a Chi-square distribution (p value <0.05). For genes that show evidence for adaptive evolution, positive selection at individual sites (dN/dS) were computed using Bayes Empirical Bayes (PAML) and sites with posterior probabilities >95% were called significant. Furthermore, dN/dS for every gene were also estimated using a conservative counting based algorithm, SLAC(23) from HyPhy wherever applicable depending on the dataset size.

Although codon based models are robust for demographic influences and could detect both ancient and multiple recurring selective sweeps, population genetics methods were known to be more sensitive to detect ongoing or relatively recent sweep events. Therefore, we employed neutrality tests including Tajima's D(24), Fu&Li's D* and Fu&Li's F*(25) (for intra-species) and Fu's Fs(26) (DNASP version 5) that rely on the underlying allele frequency spectrum to look for excess of rare alleles that might consequently arise, neighboring recently fixed alleles or those which are close to fixation. This phenomenon is a result of linkage of neutrally evolving alleles to fitness conferring alleles that reduce the recombination rate in the vicinity. To detect such genomic regions under selection, a sliding window approach was used wherein a whole genome alignment of relevant isolates was split into overlapping windows of constant size (e.g., window size 1 kb; step size 0.5 kb) and every region was evaluated for the given test statistic independently. Windows that constitute the top 1% negative rank ordered test statistic were called significant. Ranking regions under selection from a genomic distribution of ranks has been previous reported(27). Furthermore, these tests were repeated for multiple window sizes (2 kb, 5, 10 and 20 kb windows) in order to identify genomic regions that are under selection independent of the window size used. In addition to neutrality tests, two other tests that are sensitive to detect recent selective sweep events on whole genomes were used; 1. SweeD (i.e., Sweep Detector(28)), is an extension of SweepFinder(29), based on composite likelihood and rely on empirical site frequency spectrum of the dataset. The strength of a selective sweep is evaluated using the test statistic alpha (α=rln(2N)/S; r=recombination rate, N is population size and S is selection coefficient) wherein positions in the genomic alignment that maximizes the likelihood for alpha within a chosen window (1 kb, 2, 5, 10 and 20 kb) were regarded as the likely region of sweep in the given window and likewise the procedure was iterated over the entire genome alignment (concatenated CDS alignments). Windows that constitute the top 1% rank ordered alpha statistic were considered significant. 2. Omega statistic implementation OmegaPlus(30) is based on the observation that average linkage disequilibrium (LD) between SNP pairs, either on the left or on the right side of a potentially selective SNP, should be greater than the average LD values between SNP pairs across the beneficial mutation. An exhaustive scan of sub-genomic regions (RMAX) was performed by evaluating the omega statistics for all possible sub-regions within RMAX and the position with greatest omega, i.e., likely position of the sweep was reported. Sub-regions within RMAX were defined by minimum ( 1/10th of RMAX) and maximum window sizes (same as RMAX) respectively. The scan was iterated over the entire genome alignment and top windows that cover the most likely sweep positions were estimated using rank ordering regions by omega. Furthermore, the procedure was iterated for many different RMAX values (window sizes as in SweeD). Finally, all population genetics tests described above were applied to 4 separate but overlapping datasets as described in FIGS. 15A and 15B.

### REFERENCES

1. Morens D M & Fauci A S (2013) Emerging infectious diseases: threats to human health and global stability. PLoS pathogens 9(7):e1003467.
2. Rohde H, et al. (2011) Open-source genomic analysis of Shiga-toxin-producing E. coli 0104:H4. The New England journal of medicine 365(8):718-724.
3. Petty N K, et al. (2014) Global dissemination of a multidrug resistant Escherichia coli clone. Proceedings of the National Academy of Sciences of the United States of America 111(15):5694-5699.
4. Li M, et al. (2012) MRSA epidemic linked to a quickly spreading colonization and virulence determinant. Nature medicine 18(5):816-819.
5. Davies M R, et al. (2015) Emergence of scarlet fever Streptococcus pyogenes emm12 clones in Hong Kong is associated with toxin acquisition and multidrug resistance. Nature genetics 47(1):84-87.
6. He M, et al. (2013) Emergence and global spread of epidemic healthcare-associated Clostridium difficile. Nature genetics 45(1):109-113.
7. Nicolas-Chanoine M H, et al. (2008) Intercontinental emergence of Escherichia coli clone 025:H4-ST131 producing CTX-M-15. J Antimicrob Chemother 61(2):273-281.
8. Wilson D J (2012) Insights from genomics into bacterial pathogen populations. PLoS pathogens 8(9):e1002874.
9. Ruiz-Palacios G M (2007) The health burden of Campylobacter infection and the impact of antimicrobial resistance: playing chicken. Clinical infectious diseases: an official publication of the Infectious Diseases Society of America 44(5):701-703.
10. Scallan E, et al. (2011) Foodborne illness acquired in the United States—major pathogens. Emerging infectious diseases 17(1):7-15.
11. Rotariu O, et al. (2009) Spatiotemporal homogeneity of Campylobacter subtypes from cattle and sheep across northeastern and southwestern Scotland. Appl Environ Microbiol 75(19):6275-6281.
12. Hermans D, et al. (2012) Poultry as a host for the zoonotic pathogen Campylobacter jejuni. Vector borne and zoonotic diseases 12(2):89-98.
13. Kaakoush N O, Castano-Rodriguez N, Mitchell H M, & Man S M (2015) Global Epidemiology of Campylobacter Infection. Clinical microbiology reviews 28(3):687-720.
14. Mannering S A, West D M, Fenwick S G, Marchant R M, & O'Connell K (2006) Pulsed-field gel electrophoresis of Campylobacter jejuni sheep abortion isolates. Veterinary microbiology 115(1-3):237-242.
15. Sahin O, et al. (2008) Emergence of a tetracycline-resistant Campylobacter jejuni clone associated with outbreaks of ovine abortion in the United States. Journal of clinical microbiology 46(5):1663-1671.
16. Sahin O, et al. (2012) Molecular evidence for zoonotic transmission of an emergent, highly pathogenic Campylobacter jejuni clone in the United States. Journal of clinical microbiology 50(3):680-687.
17. Burrough E R, Sahin O, Plummer P J, Zhang Q, & Yaeger M J (2009) Pathogenicity of an emergent, ovine abortifacient Campylobacter jejuni clone orally inoculated into pregnant guinea pigs. American journal of veterinary research 70(10):1269-1276.
18. Wu Z, et al. (2013) Multi-omics approaches to deciphering a hypervirulent strain of Campylobacter jejuni. Genome biology and evolution 5(11):2217-2230.
19. Parkhill J, et al. (2000) The genome sequence of the food-borne pathogen Campylobacter jejuni reveals hypervariable sequences. Nature 403(6770):665-668.
20. Mandavi J, et al. (2014) A novel O-linked glycan modulates Campylobacter jejuni major outer membrane protein-mediated adhesion to human histo-blood group antigens and chicken colonization. Open biology 4:130202.
21. De E, et al. (2000) MOMP (major outer membrane protein) of Campylobacter jejuni; a versatile pore-forming protein. FEBS letters 469(1):93-97.
22. Moser I, Schroeder W, & Salnikow J (1997) Campylobacter jejuni major outer membrane protein and a 59-kDa protein are involved in binding to fibronectin and INT 407 cell membranes. FEMS microbiology letters 157(2):233-238.
23. Zhang Q, Meitzler J C, Huang S, & Morishita T (2000) Sequence polymorphism, predicted secondary structures, and surface-exposed conformational epitopes of Campylobacter major outer membrane protein. Infect Immun 68(10):5679-5689.
24. Wilson D J, et al. (2009) Rapid evolution and the importance of recombination to the gastroenteric pathogen Campylobacter jejuni. Mol Biol Evol 26(2):385-397.
25. Sheppard S K & Maiden M C (2015) The evolution of Campylobacter jejuni and Campylobacter coli. Cold Spring Harbor perspectives in biology 7(8):a018119.
26. Bentley S D & Parkhill J (2015) Genomic perspectives on the evolution and spread of bacterial pathogens. Proceedings. Biological sciences/The Royal Society 282(1821).
27. Alachiotis N, Stamatakis A, & Pavlidis P (2012) OmegaPlus: a scalable tool for rapid detection of selective sweeps in whole-genome datasets. Bioinformatics 28(17):2274-2275.
28. Pavlidis P, Zivkovic D, Stamatakis A, & Alachiotis N (2013) SweeD: likelihood-based detection of selective sweeps in thousands of genomes. Mol Biol Evol 30(9):2224-2234.
29. Fu Y X & Li W H (1993) Statistical tests of neutrality of mutations. Genetics 133(3):693-709.
30. Fu Y X (1997) Statistical tests of neutrality of mutations against population growth, hitchhiking and background selection. Genetics 147(2):915-925.
31. Nielsen R (2001) Statistical tests of selective neutrality in the age of genomics. Heredity 86(Pt 6):641-647.
32. Messer P W & Neher R A (Estimating the strength of selective sweeps from deep population diversity data. Genetics 191(2):593-605.
33. Oleksyk T K, Smith M W, & O'Brien S J (Genome-wide scans for footprints of natural selection. Philos Trans R Soc Lond B Biol Sci 365(1537):185-205.
34. Ramirez-Soriano A, Ramos-Onsins S E, Rozas J, Calafell F, & Navarro A (2008) Statistical power analysis of neutrality tests under demographic expansions, contractions and bottlenecks with recombination. Genetics 179(1):555-567.
35. Ramos-Onsins S E & Rozas J (2002) Statistical properties of new neutrality tests against population growth. Mol Biol Evol 19(12):2092-2100.
36. Nasser W, et al. (2014) Evolutionary pathway to increased virulence and epidemic group A Streptococcus disease derived from 3,615 genome sequences. Proceedings of the National Academy of Sciences of the United States of America 111(17):E1768-1776.
37. Viana D, et al. (2015) A single natural nucleotide mutation alters bacterial pathogen host tropism. Nature genetics 47(4):361-366.
38. Brault A C, et al. (2007) A single positively selected West Nile viral mutation confers increased virogenesis in American crows. Nature genetics 39(9):1162-1166.
39. Sanson M, et al. (2015) A naturally occurring single amino acid replacement in multiple gene regulator of group A Streptococcus significantly increases virulence. The American journal of pathology 185(2):462-471.
40. Carroll R K, et al. (2011) Naturally occurring single amino acid replacements in a regulatory protein alter streptococcal gene expression and virulence in mice. The Journal of clinical investigation 121(5):1956-1968.
41. Bagos P G, Liakopoulos T D, Spyropoulos I C, & Hamodrakas S J (2004) PRED-TMBB: a web server for predicting the topology of beta-barrel outer membrane proteins. Nucleic acids research 32 (Web Server issue):W400-404.
42. Spyropoulos I C, Liakopoulos T D, Bagos P G, & Hamodrakas S J (2004) TMRPres2D: high quality visual representation of transmembrane protein models. Bioinformatics 20(17):3258-3260.

TABLE A
Strains in study and reference genomes

| | | | | | | | | Accession |
|---|---|---|---|---|---|---|---|---|
| | Strain Name | Host/Disease | Sources | Region | Year | CloneSA* | ST | No. |
| strains sequenced | CA6e | Sheep/Abortion | Aborted placenta | CA, USA | 1991 | Yes | 8 | SRR3094400 |
| in this study | CA7e | Sheep/Abortion | Aborted placenta | CA, USA | 1992 | Yes | 8 | SRR3094401 |
| | ID1 | Sheep/Abortion | Aborted placenta | ID, USA | 1992 | Yes | 8 | SRR3094402 |
| | ID7 | Sheep/Abortion | Aborted placenta | ID, USA | 1992 | Yes | 8 | SRR3094489 |
| | CA8e | Sheep/Abortion | Aborted placenta | CA, USA | 1993 | Yes | 8 | SRR3094403 |
| | ID12 | Sheep/Abortion | Aborted placenta | ID, USA | 1993 | Yes | 8 | SRR3094404 |
| | ID15 | Sheep/Abortion | Aborted placenta | ID, USA | 1993 | Yes | 8 | SRR3094405 |
| | ID28 | Sheep/Abortion | Aborted placenta | ID, USA | 1993 | Yes | 8 | SRR3094406 |
| | ID31 | Sheep/Abortion | Aborted placenta | ID, USA | 1993 | Yes | 8 | SRR3094407 |
| | ID34 | Sheep/Abortion | Aborted placenta | ID, USA | 1993 | Yes | 8 | SRR3094408 |
| | ID27 | Sheep/Abortion | Aborted placenta | ID, USA | 1993 | Yes | 8 | SRR3094490 |
| | CA3e | Sheep/Abortion | Aborted placenta | CA, USA | 1999 | Yes | 8 | SRR3094409 |
| | CA4e | Sheep/Abortion | Aborted placenta | CA, USA | 1999 | Yes | 8 | SRR3094410 |
| | CA5e | Sheep/Abortion | Aborted placenta | CA, USA | 2000 | Yes | 8 | SRR3094411 |
| | CA7 | Sheep/Abortion | Aborted placenta | CA, USA | 2003 | Yes | 8 | SRR3094414 |
| | CA10 | Cattle/Abortion | Aborted placenta | CA, USA | 2003 | Yes | 8 | SRR3094454 |
| | CA12 | Cattle/Abortion | Aborted placenta | CA, USA | 2003 | Yes | 8 | SRR3094455 |
| | VDL705 | Sheep/Abortion | Aborted placenta | IA, USA | 2003 | Yes | 8 | SRR3094412 |
| | VLD698 | Sheep/Abortion | Aborted placenta | IA, USA | 2003 | Yes | 8 | SRR3094413 |
| | D6249 | Human/Gastroenteritis | Feces | VT, USA | 2003 | Yes | 8 | SRR3094467 |
| | CA5 | Sheep/Abortion | Aborted placenta | CA, USA | 2004 | Yes | 8 | SRR3094419 |
| | CA8 | Sheep/Abortion | Aborted placenta | CA, USA | 2004 | Yes | 8 | SRR3094420 |
| | CA11 | Cattle/Abortion | Aborted placenta | CA, USA | 2004 | Yes | 8 | SRR3094456 |
| | VDL5908 | Sheep/Abortion | Aborted placenta | IA, USA | 2004 | Yes | 8 | SRR3094417 |
| | VDL3842 | Sheep/Abortion | Aborted placenta | IA, USA | 2004 | Yes | 8 | SRR3094418 |
| | ID181077-B | Sheep/Abortion | Aborted placenta | ID, USA | 2004 | Yes | 8 | SRR3094415 |
| | ID323147-F | Sheep/Abortion | Aborted placenta | ID, USA | 2004 | Yes | 8 | SRR3094416 |
| | FDAN337 | Chicken | Meat | MN, USA | 2004 | Yes | 8 | SRR3094474 |
| | FDAN342 | Chicken | Meat | MN, USA | 2004 | Yes | 8 | SRR3094475 |
| | CA3 | Sheep/Abortion | Aborted placenta | CA, USA | 2005 | Yes | 8 | SRR3094424 |
| | VDL3080 | Sheep/Abortion | Aborted placenta | IA, USA | 2005 | Yes | 8 | SRR3094422 |
| | VDL2738 | Goat/Abortion | Aborted placenta | IA, USA | 2005 | Yes | 8 | SRR3094459 |
| | VDL2918 | Sheep/Abortion | Aborted placenta | IA, USA | 2005 | Yes | 8 | SRR3094494 |
| | ID219083-A | Sheep/Abortion | Aborted placenta | ID, USA | 2005 | Yes | 8 | SRR3094421 |
| | ND3 | Cattle/Abortion | Aborted placenta | ND, USA | 2005 | Yes | 8 | SRR3094457 |
| | SD3831 | Sheep/Abortion | Aborted placenta | SD, USA | 2005 | Yes | 8 | SRR3094423 |
| | SD4165 | Sheep/Abortion | Aborted placenta | SD, USA | 2005 | Yes | 8 | SRR3094492 |
| | ID273138-G | Sheep/Abortion | Aborted placenta | ID, USA | 2006 | Yes | 8 | SRR3094425 |
| | ID428211-A | Sheep/Abortion | Aborted placenta | ID, USA | 2006 | Yes | 8 | SRR3094426 |
| | VDL6220 | Sheep/Abortion | Aborted placenta | IA, USA | 2007 | Yes | 8 | SRR3094428 |
| | ID147052-A | Sheep/Abortion | Aborted placenta | ID, USA | 2007 | Yes | 8 | SRR3094427 |
| | ND6 | Sheep/Abortion | Aborted placenta | ND, USA | 2007 | Yes | 8 | SRR3094429 |
| | VDL2192 | Sheep/Abortion | Aborted placenta | IA, USA | 2008 | Yes | 8 | SRR3094430 |
| | VDL1625 | Goat/Abortion | Aborted placenta | IA, USA | 2008 | Yes | 8 | SRR3094460 |
| | 1E2B2a | Sheep | Bile | IA, USA | 2008 | Yes | 8 | SRR3094463 |
| | 1L1F1a | Sheep | Feces | IA, USA | 2008 | Yes | 8 | SRR3094464 |
| | 2L5B2 | Sheep | Bile | IA, USA | 2008 | Yes | 8 | SRR3094465 |
| | 2L5B4 | Sheep | Bile | IA, USA | 2008 | Yes | 8 | SRR3094466 |
| | 2L5F9 | Sheep | Feces | IA, USA | 2008 | Yes | 8 | SRR3094495 |
| | ND9 | Sheep/Abortion | Aborted placenta | ND, USA | 2008 | Yes | 8 | SRR3094448 |
| | D7333 | Cow | Milk | PA, USA | 2008 | Yes | 8 | SRR3094468 |
| | D7324 | Human/Gastroenteritis | Feces | PA, USA | 2008 | Yes | 8 | SRR3094469 |
| | D7365 | Human/Gastroenteritis | Feces | RI, USA | 2008 | Yes | 8 | SRR3094470 |
| | VDL8958 | Sheep/Abortion | Aborted placenta | IA, USA | 2009 | Yes | 8 | SRR3094431 |
| | VDL35044 | Sheep/Abortion | Aborted placenta | IA, USA | 2009 | Yes | 8 | SRR3094432 |
| | VDL31248 | Cattle/Abortion | Aborted placenta | 1A, USA | 2009 | Yes | 8 | SRR3094458 |
| | VDL1957 | Sheep/Abortion | Aborted placenta | IA, USA | 2010 | Yes | 8 | SRR3094433 |
| | VDL2764 | Sheep/Abortion | Aborted placenta | IA, USA | 2010 | Yes | 8 | SRR3094434 |
| | VDL4023 | Goat/Abortion | Aborted placenta | IA, USA | 2010 | Yes | 8 | SRR3094461 |
| | VDL4350 | Goat/Abortion | Aborted placenta | IA, USA | 2010 | Yes | 8 | SRR3094462 |
| | VDL2847 | Sheep/Abortion | Aborted placenta | IA, USA | 2010 | Yes | 8 | SRR3094493 |
| | D8347 | Human/Gastroenteritis | Feces | MT, USA | 2010 | Yes | 8 | SRR3094471 |
| | CO351 | Sheep/Abortion | Aborted placenta | CO, USA | 2011 | Yes | 8 | SRR3094436 |
| | VDL5414 | Sheep/Abortion | Aborted placenta | IA, USA | 2011 | Yes | 8 | SRR3094435 |
| | VDL3452 | Sheep/Abortion | Aborted placenta | IA, USA | 2013 | Yes | 8 | SRR3094481 |
| | VDL6794 | Sheep/Abortion | Aborted placenta | IA, USA | 2013 | Yes | 8 | SRR3094482 |
| | 302 | Sheep/Abortion | Aborted placenta | IA, USA | 2014 | Yes | 8 | SRR3094496 |
| | 3008 | Sheep/Abortion | Aborted placenta | IA, USA | 2014 | Yes | 8 | SRR3094497 |
| | 5406 | Sheep/Abortion | Aborted placenta | MI, USA | 2014 | Yes | 8 | SRR3094498 |
| | FDA17848 | Human/Gastroenteritis | Feces | IA, USA | Unknown | Yes | 8 | SRR3094472 |
| | FDA17817 | Human/Gastroenteritis | Feces | IA, USA | Unknown | Yes | 8 | SRR3094473 |
| | ID25 | Sheep/Abortion | Aborted placenta | ID, USA | 1993 | Yes | 21 | SRR3094491 |
| | UK29 | Sheep/Abortion | Aborted placenta | UK | 2005 | No | 21 | SRR3094477 |
| | VDL6069 | Sheep/Abortion | Aborted placenta | IA, USA | 2010 | No | 38 | SRR3094453 |
| | UK32 | Sheep/Abortion | Aborted placenta | UK | 2005 | No | 42 | SRR3094487 |
| | ND7 | Sheep/Abortion | Aborted placenta | ND, USA | 2007 | No | 42 | SRR3094447 |
| | VDL2401 | Sheep/Abortion | Aborted placenta | IA, USA | 2007 | No | 43 | SRR3094446 |
| | UK24 | Sheep/Abortion | Aborted placenta | UK | 2005 | No | 45 | SRR3094486 |
| | VDL2945 | Sheep/Abortion | Aborted placenta | IA, USA | 2009 | No | 45 | SRR3094452 |
| | ID8 | Sheep/Abortion | Aborted placenta | ID, USA | 1993 | No | 50 | SRR3094438 |
| | ID30 | Sheep/Abortion | Aborted placenta | ID, USA | 1993 | No | 50 | SRR3094439 |
| | ID10 | Sheep/Abortion | Aborted placenta | ID, USA | 1993 | No | 50 | SRR3094440 |
| | CA1e | Sheep/Abortion | Aborted placenta | CA, USA | 1998 | No | 50 | SRR3094441 |
| | CA2e | Sheep/Abortion | Aborted placenta | CA, USA | 1998 | No | 50 | SRR3094442 |
| | ID017002-A | Sheep/Abortion | Aborted placenta | ID, USA | 2004 | No | 50 | SRR3094443 |
| | UK19 | Sheep/Abortion | Aborted placenta | UK | 2004 | No | 50 | SRR3094478 |
| | OF48 | Sheep | Feces | IA, USA | 2006 | No | 61 | SRR3094479 |
| | UK10 | Sheep/Abortion | Aborted placenta | UK | 2003 | No | 206 | SRR3094485 |
| | UK11 | Sheep/Abortion | Aborted placenta | UK | 2003 | No | 227 | SRR3094484 |
| | UK33 | Sheep/Abortion | Aborted placenta | UK | 2006 | No | 227 | SRR3094476 |
| | UK40 | Sheep/Abortion | Aborted placenta | UK | 2008 | No | 227 | SRR3094483 |
| | UK37 | Sheep/Abortion | Aborted placenta | UK | 2007 | No | 270 | SRR3094488 |
| | ID33 | Sheep/Abortion | Aborted placenta | ID, USA | 1993 | No | 441 | SRR3094437 |
| | CA2 | Sheep/Abortion | Aborted placenta | CA, USA | 2003 | No | 607 | SRR3094444 |
| | VDL4646 | Sheep/Abortion | Aborted placenta | IA, USA | 2004 | No | 806 | SRR3094445 |
| | VDL213 | Sheep/Abortion | Aborted placenta | IA.USA | 2009 | No | 806 | SRR3094451 |
| | VDL902 | Sheep/Abortion | Aborted placenta | IA, USA | 2008 | No | 982 | SRR3094449 |
| | VDL35490 | Sheep/Abortion | Aborted placenta | IA, USA | 2012 | No | 982 | SRR3094480 |
| | VDL2019 | Sheep/Abortion | Aborted placenta | IA, USA | 2008 | No | 5189 | SRR3094450 |
| Reference strains | IA3902 | Sheep/Abortion | Aborted placenta | IA, USA | 2006 | Yes | 8 | NC_017279 |
| | RM1221 | Chicken | | | | | | NC_003912 |
| | 269.97 | Human | | | | | | NC_009707 |
| | 81-176 | Human | | | | | | NC_008787 |
| | 81116 | Human | | | | | | NC_009839 |
| | NCTC11168 | Human | | | | | | NC_002163 |
| | M1 | Human | | | | | | NC_017280 |
| | 4031 | Human | | | | | | NC_022529 |
| | ICDCCJ07001 | Human | | | | | | NC_014802 |
| | S3 | Human | | | | | | NC_017281 |
| | PT14 | Human | | | | | | NC_018709 |
| | 00-2425 | Human | | | | | | NC_022362 |
| | 00-2538 | Human | | | | | | NC_022351 |
| | 00-2544 | Human | | | | | | NC_022353 |
| | 00-2426 | Human | | | | | | NC_022352 |
| | CVM_N29710 | C. coli used as outgroup | | | | | | NC_022347 |
| Transformants from | 36U-4 | Guinea pig/abortion | Uterus | | | | | SRR3094499 |
| sexual genetics study | 36U-5 | Guinea pig/abortion | Uterus | | | | | SRR3094500 |
| | 36P-2 | Guinea pig/abortion | Placenta | | | | | SRR3094501 |
| | 36P-3 | Guinea pig/abortion | Placenta | | | | | SRR3094502 |
| | 37U-2 | Guinea pig/abortion | Uterus | | | | | SRR3094503 |
| | 37U-4 | Guinea pig/abortion | Uterus | | | | | SRR3094504 |
| | 37P-1 | Guinea pig/abortion | Placenta | | | | | SRR3094505 |
| | 37P-3 | Guinea pig/abortion | Placenta | | | | | SRR3094506 |
| | 40U-2 | Guinea pig/abortion | Uterus | | | | | SRR3094507 |
| | 40U-4 | Guinea pig/abortion | Uterus | | | | | SRR3094508 |
| | 40P-1 | Guinea pig/abortion | Placenta | | | | | SRR3094509 |
| | 40P-3 | Guinea pig/abortion | Placenta | | | | | SRR3094510 |
| | NCTC11168_reseq | resequenced for control | | | | | | SRR3094511 |
| | Transformants_pool | Guinea pig/abortion | | | | | | SRR3094512 |

*Clone SA is re-defined based on whole genome phylogeny (FIG. 1a) in this study. All the ST-8 isolates cluster together as expected; only one strain ID25, which belongs to ST-21 in the same clonal complex (CC21) as ST-8, has highly similar genome to ST-8 isolates and is assigned to Clone SA accordingly.

TABLE B
Primers used in this study and PCR conditions

| | |
|---|---|
| porAF | 5′-ATGAAACTAGTTAAACTTAGTTTAGTTGCAGCTC-3′ |
| | (SEQ ID NO: 6) |
| porAR | 5′-CGCGGATCCAAGCGTTTTAGCTTGG-3′ |
| | (SEQ ID NO: 7) |
| dnaJF | 5′-GAACTGCAGTAGGAAAATTAAGACTTAAACC-3′ |
| | (SEQ ID NO: 8) |
| dnaJR | 5′-GGTAATATCTTAAAAAATGGTACTAGAGGGGATATG-3′ |
| | (SEQ ID NO: 9) |
| kanF | 5′-CGCGGATCCCGCTTATCAATATATCTATAGAATGG-3′ |
| | (SEQ ID NO: 10) |
| KanR | 5′-GAACTGCAGGATAATGCTAAGACAATCACTAAAG-3′ |
| | (SEQ ID NO: 11) |

PCR conditions
98° C. 1 min

| | | |
|---|---|---|
| 98° C. 10 sec | | |
| 60° C. 30 sec | {close oversize brace} | 35 cycles |
| 72° C. 1-3 min | | |

72° C. 10 min
4° C. ∞

Supplementary Results

Evaluation of Methods to Identify porA as the Cause of Abortion from Sequence Analysis

As discussed in the main text, knowledge that allelic variation in porA is responsible for abortion provides a critical data set by which various sequence analysis methods may be evaluated. Published data on clone SA (1, 31) led us to expect that whatever locus is driving the abortion phenotype would confer a fitness advantage and thus be under positive selection and drive a selective sweep. Furthermore, the rapid expansion of clone SA throughout the United States, and its clonality, implied that clone SA would be evolving under a population expansion model. We therefore tested methods designed to detect positive selection, selective sweeps, and recent population expansion. We performed all methods using the entire data set (58 clone SA abortion isolates; 14 clone SA other; 27 sporadic abortion isolates; 15 full genomes including IA3902 and NCTC11168) as well as several subsets of the data (FIG. 15A) to acknowledge the biased sampling, and to account for what appeared to be recombination/horizontal transfer at the porA locus. All methods were tested using concatenated orthologs that were present in all isolates; the ordering of the orthologs was preserved relative to the IA3902 genome sequence (including reverse complementation of genes encoded on the negative strand). The general strategy was to take sliding windows of the data, calculate summary statistics, and test whether windows encompassing porA had outstanding values across the genome, defined as the top 1% of windows. Sliding windows were taken as fixed windows of 1000 up to 10,000 nucleotides with a step size of half the window size. The data sets used and summarized results are shown in FIG. 15.

For selective sweeps, we evaluated SweeD and OmegaPlus(32, 33). For none of the data sets or window sizes was porA associated with extreme scores indicating a selective sweep. In fact, in many cases, porA was close to the genome median or below (i.e. less evidence for a selective sweep than the median window).

For positive selection, we used the PAML program, which uses a maximum likelihood approach, and the SLAC method from HYPHY, which tests for selection using a mutation counting method. For PAML, we set a cutoff of P<0.05 using a likelihood ratio test (LRT) and ranked the genes passing this cutoff based on dN/dS. For SLAC, we considered all significant genes without further ranking. For both of these methods, recombination detection was done using GARD from the HYPHY package, and those genes whose multiple sequence alignments showed evidence for recombination (uncorrected P-value <0.05) were split at the predicted recombination break points, and the individual fragments were analyzed separately. In all cases, porA showed significant evidence for selection. In particular, amino acids within loop 4 of porA were under positive selection in all cases. Furthermore, the amino acid differences between the loop 4 sequences of porA from NCTC11168 and IA3902 (which we showed were sufficient for conferring abortion when transferred into NCTC11168) are 172E, 181K, 183T, 188Q, 189K, 190A, 194Q, 195A and 198L. Of these, 172E and 190A were found to be under positive selection when we analyzed either the full data set or any other subset including both clone SA-containing and non-clone SA containing strain sets. To further verify that positive selection in loop 4 of porA was not specifically under selection among clone SA strains, we randomly sub-sampled (iteration=100) porA datasets from a population dataset of Campylobacter allele sequences obtained from Campylobacter locus/sequence definitions database (http://pubmlst.org/perl/bigsdb/bigsdb.pl?db=pubmlst_campylobacter_seqdef), and found that loop 4 of porA was also predicted to be evolving under positive selection in 14% of simulations. In addition to these loop 4 residues, other amino acids within porA were frequently found to be under positive selection in these tests, mostly concentrated in the extracellular loops, suggesting that positive selection might be acting on porA because some of its amino acids are surface exposed. In support of this, we also found several other outer membrane proteins (flagellar protein subunits) that were consistently predicted to be under positive selection across all of our strain subsets.

We used the following neutrality tests to test for mutation patterns consistent with selective sweep/population expansion: Tajima's D, Fu & Li's D*, Fu & Li's F*, and Fu's Fₛ. When the entire set of strains or all clone SA strains (sets (i) and (ii) in Supplementary FIG. 15a) were analyzed, none of these tests identified porA among the top 10% of genomic windows, regardless of window size. Analysis of only the clone SA isolates that were definitively isolated from abortion tissue (set (iii) in FIG. 15A) resulted in porA being detected among the top 2% of windows using Tajima's D, Fu & Li's D*, and Fu & Li's F*. while the values of Fu's Fₛ for porA windows were close to the genome median. As noted above, the GARD algorithm from HYPHY did not detect any evidence of recombination in the porA gene alignment for this subset of strains; however, manual inspection of the data revealed that one strain, CA12, appeared to have undergone recombination at porA, as the genomic phylogeny placed it definitively within clone SA (FIG. 1A) but its porA allele was highly hypervirulent (FIG. 14). Recombination can in some cases enhance the power of these population genetics statistics (34), but the effect is complicated and most simulations tend to assume no recombination occurs(35). We thus repeated the analysis excluding CA-12, which left no obvious recombination by manual inspection or by any recombination detection algorithm; this led to porA windows now being near the median for Tajima's D, Fu & Li's D*, and Fu & Li's F*. In contrast, windows overlapping porA, and specifically those overlapping loop 4 of porA, had the most extreme negative values of Fu's Fₛ (indicative of population expansion and/or selective sweep). As discussed in the main text (FIGS. 4B and 4C), the pattern of mutation surrounding loop 4 of porA was consistent with theoretical expectations of rapid population expansion subsequent to a hard selective sweep. In particular, the expectation of a recent selective sweep would be a reduction in diversity surrounding the selected allele. During the sweep, recent (and therefore rare) mutations would arise linked to this selected allele(36) (37). This overall should result in a star-like phylogeny, with an excess of rare (singleton) mutations, that are associated with a higher number of haplotypes than expected given the number of segregating sites (26). As seen in FIG. 14, porA alleles from clone SA isolates do seem to have a star-like phylogeny. There is a mild excess of rare (singleton) mutations closely linked to loop 4 of porA (FIG. 4C) when assessed by absolute number of singleton mutations (FIG. 15c). Furthermore, the total number of haplotypes in the two genomic windows containing loop 4 of porA are among the highest genome wide (FIG. 4C, FIG. 15D), despite the only mild enrichment of segregating sites and singleton mutations in these windows. Accordingly, Fu's Fₛ captures a signal related to the number of haplotypes compared with the number of segregating sites, integrating all this data. As shown in FIG. 15D, the combination of high haplotypes relative to segregating sites is indeed extreme for porA.

While in general the locus of interest is not known, we here have tested whether known methods under any condition are able to detect porA. Given that we knew that porA was the locus responsible for abortion, the manual nature of this analysis to find one test that ranked porA highly cannot be construed as a general method by which genomic analysis should be used to identify a causative disease allele such as porA. However, this analysis indicates that strong signals, similar to those predicted by theory, do exist in real data sets. Our results indicate that recombination, strain sampling strategies, and the quality of metadata/phenotype information can have a large impact on the ability of current tests to detect such a signal (Fu's Fₛ is non-significant when we include one strain that carries a hypervirulent, recombined allele); and on the occurrence of false positive results (Tajima's D, Fu & Li's D*, and Fu & Li's F* all appear to be detecting a recombination event in porA in data set (iii) instead of a true signal of population expansion/selective sweep). However, the performance of Fu's Fₛ (generally the most powerful for detecting population expansion but one of the most sensitive to recombination) matches previous simulation studies(34, 38). Therefore, these results suggest that further work to enhance the power for detecting recombination may be valuable for analysis of other large genomic data sets. In addition, simulations studies are effective methods for evaluating such population genetic tests, as data sets such as ours are still exceedingly rare.

### SUPPLEMENTARY REFERENCES

1. Sahin O, et al. (2008) Emergence of a tetracycline-resistant Campylobacter jejuni clone associated with outbreaks of ovine abortion in the United States. Journal of clinical microbiology 46(5):1663-1671.
2. Sahin O, et al. (2012) Molecular evidence for zoonotic transmission of an emergent, highly pathogenic Campylobacter jejuni clone in the United States. Journal of clinical microbiology 50(3):680-687.
3. Burrough E R, Sahin O, Plummer P J, Zhang Q, & Yaeger M J (2009) Pathogenicity of an emergent, ovine abortifacient Campylobacter jejuni clone orally inoculated into pregnant guinea pigs. American journal of veterinary research 70(10):1269-1276.
4. Wu Z, et al. (2013) Multi-omics approaches to deciphering a hypervirulent strain of Campylobacter jejuni. Genome biology and evolution 5(11):2217-2230.
5. Parkhill J, et al. (2000) The genome sequence of the food-borne pathogen Campylobacter jejuni reveals hypervariable sequences. Nature 403(6770):665-668.
6. Li H, et al. (2009) The Sequence Alignment/Map format and SAMtools. Bioinformatics 25(16):2078-2079.
7. Li H & Durbin R (2010) Fast and accurate long-read alignment with Burrows-Wheeler transform. Bioinformatics 26(5):589-595.
8. Kurtz S, et al. (2004) Versatile and open software for comparing large genomes. Genome biology 5(2):R12.
9. Price M N, Dehal P S, & Arkin A P (2010) FastTree 2—approximately maximum-likelihood trees for large alignments. PloS one 5(3):e9490.
10. Marttinen P, et al. (2012) Detection of recombination events in bacterial genomes from large population samples. Nucleic acids research 40(1):e6.
11. Zerbino D R & Birney E (2008) Velvet: algorithms for de novo short read assembly using de Bruijn graphs. Genome research 18(5):821-829.
12. Hyatt D, et al. (Prodigal: prokaryotic gene recognition and translation initiation site identification. BMC Bioinformatics 11:119.
13. Contreras-Moreira B & Vinuesa P (GET_HOMOLOGUES, a versatile software package for scalable and robust microbial pangenome analysis. Applied and environmental microbiology 79(24):7696-7701.
14. Davis L, Young K, & DiRita V (2008) Genetic manipulation of Campylobacter jejuni. Current protocols in microbiology Chapter 8:Unit 8A 2 1-8A 2 17.
15. Darling A C, Mau B, Blattner F R, & Perna N T (2004) Mauve: multiple alignment of conserved genomic sequence with rearrangements. Genome research 14(7):1394-1403.
16. Wilm A, et al. (LoFreq: a sequence-quality aware, ultra-sensitive variant caller for uncovering cell-population heterogeneity from high-throughput sequencing datasets. Nucleic acids research 40(22): 11189-11201.
17. Wosten M M, Boeve M, Koot M G, van Nuenen A C, & van der Zeijst B A (1998) Identification of Campylobacter jejuni promoter sequences. Journal of bacteriology 180(3):594-599.
18. Sievers F, et al. (Fast, scalable generation of high-quality protein multiple sequence alignments using Clustal Omega. Mol Syst Biol 7:539.
19. Librado P & Rozas J (2009) DnaSP v5: a software for comprehensive analysis of DNA polymorphism data. Bioinformatics 25(11):1451-1452.
20. Kosakovsky Pond S L, Posada D, Gravenor M B, Woelk C H, & Frost S D (2006) GARD: a genetic algorithm for recombination detection. Bioinformatics 22(24):3096-3098.
21. Pond S L, Frost S D, & Muse S V (2005) HyPhy: hypothesis testing using phylogenies. Bioinformatics 21(5):676-679.
22. Yang Z (2007) PAML 4: phylogenetic analysis by maximum likelihood. Molecular biology and evolution 24(8):1586-1591.
23. Kosakovsky Pond S L & Frost S D (2005) Not so different after all: a comparison of methods for detecting amino acid sites under selection. Molecular biology and evolution 22(5):1208-1222.
24. Tajima F (1989) Statistical method for testing the neutral mutation hypothesis by DNA polymorphism. Genetics 123(3):585-595.
25. Fu Y X & Li W H (1993) Statistical tests of neutrality of mutations. Genetics 133(3):693-709.
26. Fu Y X (1997) Statistical tests of neutrality of mutations against population growth, hitchhiking and background selection. Genetics 147(2):915-925.
27. Carlson C S, et al. (2005) Genomic regions exhibiting positive selection identified from dense genotype data. Genome research 15(11):1553-1565.
28. Pavlidis P, Zivkovic D, Stamatakis A, & Alachiotis N (SweeD: likelihood-based detection of selective sweeps in thousands of genomes. Molecular biology and evolution 30(9):2224-2234.
29. Nielsen R, et al. (2005) Genomic scans for selective sweeps using SNP data. Genome research 15(11):1566-1575.
30. Alachiotis N, Stamatakis A, & Pavlidis P (OmegaPlus: a scalable tool for rapid detection of selective sweeps in whole-genome datasets. Bioinformatics 28(17):2274-2275.
31. Wu Z, et al. (2014) Genetic diversity and antimicrobial susceptibility of Campylobacter jejuni isolates associated with sheep abortion in the United States and Great Britain. Journal of clinical microbiology 52(6):1853-1861.
32. Pavlidis P, Zivkovic D, Stamatakis A, & Alachiotis N (2013) SweeD: likelihood-based detection of selective sweeps in thousands of genomes. Molecular biology and evolution 30(9):2224-2234.
33. Alachiotis N, Stamatakis A, & Pavlidis P (2012) OmegaPlus: a scalable tool for rapid detection of selective sweeps in whole-genome datasets. Bioinformatics 28(17):2274-2275.
34. Ramirez-Soriano A, Ramos-Onsins S E, Rozas J, Calafell F, & Navarro A (2008) Statistical power analysis of neutrality tests under demographic expansions, contractions and bottlenecks with recombination. Genetics 179(1):555-567.
35. Thornton K (2005) Recombination and the properties of Tajima's D in the context of approximate-likelihood calculation. Genetics 171(4):2143-2148.
36. Messer P W & Neher R A (2012) Estimating the strength of selective sweeps from deep population diversity data. Genetics 191(2):593-605.
37. Oleksyk T K, Smith M W, & O'Brien S J (2010) Genome-wide scans for footprints of natural selection. Philos Trans R Soc Lond B Biol Sci 365(1537):185-205.
38. Ramos-Onsins S E & Rozas J (2002) Statistical properties of new neutrality tests against population growth. Molecular biology and evolution 19(12):2092-2100.

### Example 2

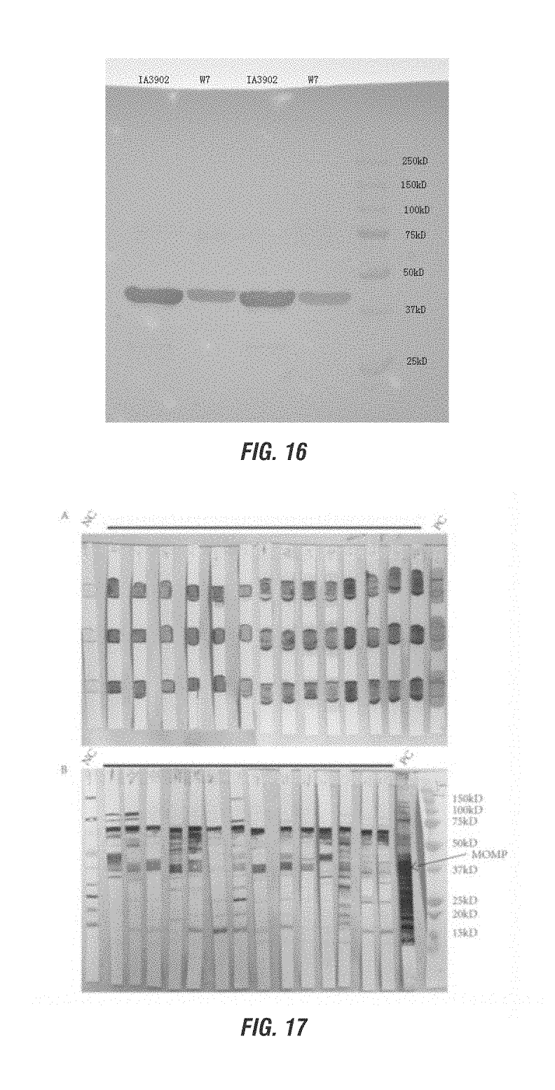

Previously, we have shown that the sequence variations in the predicted loop 4 region of the major outer membrane protein (MOMP) of Campylobacter jejuni IA3902 is important for its virulence in inducing abortion. To assess if loop 4 is immunogenic, the Loop 4 peptide (AEEQGADLLGKSTISTTQKAAPFQADSLGNL) (SEQ ID NO: 1) was synthesized and subsequently conjugated to KLH. The KLH-conjugated peptide antigen was injected subcutaneously into rabbits. The primary immunization was done with 0.5 mg peptide and then three booster immunizations were followed, each with 0.25 mg peptide on Day 14, 28, and 42, respectively, post the primary immunization. Rabbit sera were collected at Days 35 and 56, respectively. Antibody was purified by affinity purification and used for immunoblotting. As shown in FIG. 16, the rabbit anti-loop 4 antibody specifically recognized the MOMP protein in the whole lysate of C. jejuni cells. Interestingly, the reactivity of the antibody to the MOMP of strain IA3902 (a virulent strain) is stronger than to the MOMP of strain W7 (a non-virulent strain), indicating the relative specificity of the antibody toward MOMP of C. jejuni IA3902

To increase the yield of the Loop 4 peptide, the loop 4 of IA3902 (AEEQGADLLGKSTISTTQKAAPFQADSLGNL) (SEQ ID NO: 1) was concatenated by a linker (GGSSGG) (SEQ ID NO: 5) and was expressed in E. coli JM109 using plasmid pQE-1 (QIAGEN Inc, Valencia, Calif.). The expressed recombinant product contained four copies of loop 4 of MOMP and 3 copies of the linker as well as the N-terminal His leader sequence (MKHHHHHHQLHAGAHAEEQGADLLGKSTISTTQKAAPFQADSLGNLGGSSGGA EEQGADLLGKSTISTTQKAAPFQADSLGNLGGSSGGAEEQGADLLGKSTISTTQKA APFQADSLGNLGGSSGGAEEQGADLLGKSTISTTQKAAPFQADSLGNL) (SEQ ID NO: 4). Expression of the recombined peptide was induced by addition of 1 mM IPTG to the E. coli culture for 5 hours at 37° C. The recombinant peptide was purified using the Ni-NTA Superflow Column (Qiagen, Valencia, Calif., USA) under native conditions. The purified recombinant loop 4 peptide was then desalted by Pierce™ Protein Concentrators (Thermo Scientific™) and dissolved in PBS buffer.

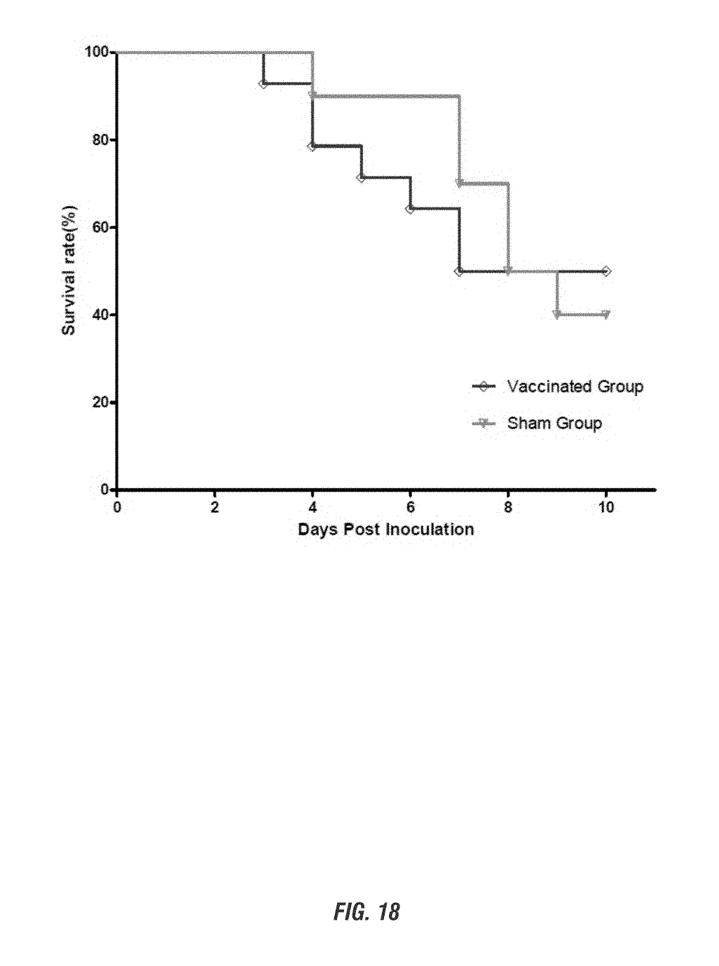

The recombinant loop 4 peptide was then mixed with an aluminum hydroxide gel adjuvant (Alhydrogel 2%, Accurate Chemical & Scientific Corp.) to prepare the loop 4 peptide vaccine. Hartley pregnant guinea pigs were obtained from a commercial source (Elm Hill Labs, Chelmsford, Mass.). Each of the guinea pigs in the vaccinated group was immunized by subcutaneous injection of 200 μl of the vaccine twice with 2 weeks apart, while pigs in the sham control group received the adjuvant only. Guinea pig sera were collected before immunization and after immunization. Two weeks after the second vaccination, guinea pigs were challenged orally with the highly virulent Campylobacter jejuni strain IA3902. Antibodies against the loop 4 peptide in the immunized guinea pigs was analyzed by dot blotting and Western blotting (FIG. 17). The results showed that the loop 4 peptide vaccine induced antibodies in the immunized guinea pigs that recognized the recombinant peptides (FIG. 17A). However, the individual guinea pig sera from the vaccine group showed varied reactivity with the MOMP protein in IA 3902 (FIG. 17B). The protection against infection was evaluated by using the abortion rates. Currently the vaccine trial is still ongoing, but the results obtained so far indicated that the peptide vaccine induced limited protection against the disease (FIG. 18). However, there are still guinea pigs that have not aborted in both the vaccinated group and the sham control group, and the complete result of the vaccine trial will be summarized when the trail is finished.

In summary, these results indicate the loop 4 peptide of MOMP is immunogenic, but formulation of vaccines based on the peptide may need to be optimized. It is possible that the conformation of the loop 4 is important for inducing protection, and concatenation in the expression of the recombinant peptide might affect the appropriate folding of the peptide. These possibilities will be examined in future experiments.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

TABLE OF SEQUENCES

| | |
|---|---|
| SEQ ID NO: 1 | Hypervirulent C. jejuni MOMP peptide |
| SEQ ID NO: 2 | C. jejuni nucleotide sequence encoding same |
| SEQ ID NO: 3 | MOMP of C. jejuni IA3902 (FIG. 3C) |
| SEQ ID NO: 4 | Four copies of Loop4 of MOMP and 3 copies of the |
| | linker and the His N-terminal leader sequence |
| SEQ ID NO: 5 | Linker sequence |
| SEQ ID NO: 6 | porAF Primer |
| SEQ ID NO: 7 | porAR Primer |
| SEQ ID NO: 8 | dnaJF Primer |
| SEQ ID NO: 9 | dnaJR Primer |
| SEQ ID NO: 10 | kanF Primer |
| SEQ ID NO: 11 | KanR Primer |

What is claimed is:

1. An immunogenic composition for protecting animals against C. jejuni, including a hypervirulent strain, the composition comprising a hypervirulent C. jejuni major outer membrane protein segment wherein the MOMP polypeptide comprises SEQ ID NO: 1 with and a carrier.

2. The composition of claim 1, wherein the composition is in the form of an inactivated, hypervirulent C. jejuni whole cell that comprises and/or expresses the C. jejuni major outer membrane polypeptide of SEQ ID NO: 1.

3. The composition of claim 1, wherein the composition is in the form of an inactivated recombinant bacterium, wherein said bacterium comprises and/or expresses the C. jejuni major outer membrane polypeptide.

4. The composition of claim 1, wherein the composition is in the form of an isolated, recombinant C. jejuni MOMP polypeptide.

5. The composition of claim 4, wherein the isolated, recombinant C. jejuni hypervirulent major outer membrane polypeptide is expressed from a vector.

6. The composition of claim 5, wherein the vector is E. coli or another bacterial organism.

7. The composition of claim 5, wherein the vector is a live or inactivated vector.

8. The composition of claim 1, wherein the C. jejuni major outer membrane polypeptide further comprises a conservatively modified variant of SEQ ID NO: 1.

9. The composition of claim 1, further comprising at least one additional antigen.

10. The composition of claim 9, wherein the at least one additional antigen is protective against a disease in animals caused by a microorganism.

11. The composition of claim 10, wherein the microorganism comprises a bacterium, virus, or protozoan.

12. The composition of claim 1, wherein the composition further comprises an adjuvant.

13. The composition of claim 12, wherein the adjuvant is selected from the group consisting of an oil-in-water adjuvant, a polymer and water adjuvant, a water-in-oil adjuvant, an aluminum hydroxide adjuvant, a vitamin E adjuvant and combinations thereof.

14. A method of immunizing a sheep against a hypervirulent C. jejuni strain, the method comprising administering to the sheep the composition of claim 1.

15. The method of claim 14, wherein the composition is administered intranasally, intramuscularly, intradermally, subcutaneously, or orally.

16. The method of claim 15, wherein the composition is administered in a single dose.

17. The method of claim 15, wherein the composition is administered as two or more doses.

18. The method of claim 15, wherein the composition is administered to animals at 3 weeks of age or older, prior to or during pregnancy.

19. A kit comprising: a bottle comprising a vaccine composition for protecting animals against a highly virulent C. jejuni strain, the composition comprising a hypervirulent C. jejuni major outer membrane polypeptide, wherein the major outer membrane polypeptide comprises SEQ ID NO: 1.

20. The kit of claim 19, wherein the composition in the bottle is provided as a ready-to-use liquid composition.

21. The kit of claim 20, wherein the composition is provided in a lyophilized form.

22. The kit of claim 21, further comprising a diluent.

23. An immunogenic composition comprising C. jejuni major outer membrane protein, and one or more excipients in an amount effective to elicit production of protective immune responses against the C. jejuni virus following administration to a subject.

24. The immunogenic composition of claim 23 wherein the major outer membrane protein is SEQ ID NO:1.

25. The immunogenic composition of claim 23 wherein the subject is a human, cow, sheep, goat, and chicken.

26. A method of producing a protective immune response against a hypervirulent C. jejuni strain in a subject comprising:
administering to the subject the immunogenic composition of claim 23 in an amount and duration effective to produce the protective immune response.

27. The method of claim 26 wherein the protective immune response reduces clinical disease (such as abortion) and/or virus reproduction in the subject.

28. The method of claim 26 wherein the protective immune response reduces bacterial shedding in the subject.

29. The method of claim 26 wherein the subject has been exposed to hypervirulent C. jejuni.

30. The method of claim 26 wherein the subject is suffering from a hypervirulent C. jejuni infection.

31. A method of detecting the presence of hypervirulent C. jejuni in sample comprising:
assaying said sample for the presence of a hypervirulent C. jejuni major outer membrane protein of the invention or a nucleic acid sequence encoding the same.

32. The method of claim 1 wherein said protein comprises SEQ ID NO:1.

33. The method of claim 31 wherein said assay is an antibody-based assay, a nucleic acid-based assay, a nucleic acid-based differential assay, or real-time RT-PCR.


SEQUENCE LISTING (11 sequences)

SEQ ID NO: 1 - protein, 31 aa, Campylobacter jejuni
AEEQGADLLGKSTISTTQKAAPFQADSLGNL

SEQ ID NO: 2 - DNA, 93 bp, Campylobacter jejuni
gcggaagagcaaggtgcagatttattaggaaaaagtactatatctacaacacagaaagcagctccttttcaagcagattcattaggaaatctt

SEQ ID NO: 3 - protein, 424 aa, Campylobacter jejuni
MKLVKLSLVAALAAGAFSAANATPLEEAIKDVDVSGVLRYRYDTGNFDKNFVNNSNLNNSKQNHKYRAQVNFSAAIADNFKAFIQFDYNAADGGYGANGIKNDQKGLFVRQLYLTYTNEDVATSVIAGKQQLNTIWTDNGVDGLVGTGIKVVNNSIDGLTLAAFAVDSFMAEEQGADLLGKSTISTTQKAAPFQADSLGNLYGAAAVGSYDLAGGQFNPQLWLAYWDQVAFFYAVDAAYSTTIFDGINWTLEGAYLGNSLDSELNDKRHANGNLFALKGSIEVNGWDASLGGLYYGDKEKASTVVIEDQGNLGSLLAGEEIFYTTGSRLNGDTGRNIFGYVTGGYTFNETVRVGADFVYGGTKTEATNHLGGGKKLEAVARVDYKYSPKLNFSAFYSYVNLDQGVNTNESADHSTVRLQALYKF

SEQ ID NO: 4 - protein, 157 aa, Artificial Sequence
  Synthetic construct
MKHHHHHHQLHAGAHAEEQGADLLGKSTISTTQKAAPFQADSLGNLGGSSGGAEEQGADLLGKSTISTTQKAAPFQADSLGNLGGSSGGAEEQGADLLGKSTISTTQKAAPFQADSLGNLGGSSGGAEEQGADLLGKSTISTTQKAAPFQADSLGNL

SEQ ID NO: 5 - protein, 6 aa, Artificial Sequence
  Linker sequence
GGSSGG

SEQ ID NO: 6 - DNA, 34 bp, Artificial Sequence
  Primer
atgaaactagttaaacttagtttagttgcagctc

SEQ ID NO: 7 - DNA, 25 bp, Artificial Sequence
  Primer
cgcggatccaagcgttttagcttgg

SEQ ID NO: 8 - DNA, 31 bp, Artificial Sequence
  Primer
gaactgcagtaggaaaattaagacttaaacc

SEQ ID NO: 9 - DNA, 36 bp, Artificial Sequence
  Primer
ggtaatatcttaaaaaatggtactagaggggatatg

SEQ ID NO: 10 - DNA, 35 bp, Artificial Sequence
  Primer
cgcggatcccgcttatcaatatatctatagaatgg

SEQ ID NO: 11 - DNA, 34 bp, Artificial Sequence
  Primer
gaactgcaggataatgctaagacaatcactaaag